(19) United States
(12) Patent Application Publication
(10) Pub. No.: US 2023/0266343 A1
(43) Pub. Date: Aug. 24, 2023

(54) Methods, Systems & Kits for Prediction, Detection, Monitoring & Treatment of Alzheimer's Disease

(71) Applicant: Baseline Bioscience, Inc., Irvine, CA (US)

(72) Inventors: Balwant Rai, Rodovre (DK); Jasdeep Kaur, Rodovre (DK)

(73) Assignee: Baseline Bioscience, Inc., Irvine, CA (US)

(21) Appl. No.: 18/140,902

(22) Filed: Apr. 28, 2023

Related U.S. Application Data
(63) Continuation of application No. PCT/US2020/058336, filed on Oct. 30, 2020.

(51) Int. Cl. G01N 33/68

(52) U.S. Cl. CPC G01N 33/6896; G01N 2333/4745; G01N 2333/95; G01N 2800/50; G01N 2800/2821; G01N 2333/70503

(57) ABSTRACT

Methods, systems, kits, and other techniques and discoveries for the prediction, detection, monitoring, treatment, and general diagnosis and prognosis of Alzheimer's disease (AD) in subjects utilizing information obtained by detection of biomarkers and biomarker combinations present in the saliva of subjects afflicted with AD. Aspects of the invention may be implemented to detect, monitor, treat and diagnose subjects who are asymptomatic of AD early in the disease process. Other aspects of the invention may be implemented to identify and differentiate the level of severity of AD in a subject such as, for example, identification of mild AD in a subject who is completely asymptomatic of AD, differentiation of mild AD from moderate AD, and differentiation of moderate AD from severe AD.

### RELATED APPLICATION

This application is a continuation of international patent application Serial No. PCT/US2020/058336 filed with the United States Patent & Trademark Office as PCT Receiving Office on Oct. 30, 2020 and designating the United States. The entire content of international patent application Serial No. PCT/US2020/058336 is incorporated herein by reference for continuity.

### FIELD

The present invention relates to the use of salivary biomarkers for evaluation of health conditions. More specifically, the present invention relates to implementation of salivary biomarkers for the prediction, detection, monitoring, treatment, and general diagnosis and prognosis of neurological disorders, such as Alzheimer's disease.

### BACKGROUND

Alzheimer's disease, also known by the acronym “AD”, is a neurodegenerative disease which is the leading cause of progressive dementia in human subjects. It is estimated that over 46.8 million people worldwide suffered from some degree of dementia as of 2015. By 2030, it is estimated that the number of people suffering from dementia worldwide will increase to approximately 74.7 million people. Dementia is most prevalent in older age groups and the anticipated increase in people suffering from dementia is due, in part, to the general aging of the population throughout the world. Martin A. Prince, “World Alzheimer Report 2015: The Global Impact of Dementia: An Analysis of Prevalence, Incidence, Cost and Trends,” Alzheimer's Disease International (2015).

AD is known to be responsible for approximately 60% to 80% of dementia-related cases in people age 65 years or older. Fortunately, AD is less prevalent in people younger than age 65. But for this younger age group, AD is still believed to account for approximately 30-40% of the dementia. A. H. Simonsen, et al., Recommendations for CSF AD Biomarkers In The Diagnostic Evaluation of Dementia, Alzheimer's & Dementia vol. 13, no. 3, 274-284 (2017). In all age groups affected by AD, cognitive deficits and accelerating neurodegenerative processes increase with the duration of the disease.

AD and the accompanying dementia is typically characterized by degree of severity as mild, moderate, or severe. The cognitive function of the patient and severity of the AD may be identified, for example, by means of what is referred to as the “Mini-Mental State Examination” (MMSE). Folstein, M. F., et al., Mini-Mental State. A Practical Method for Grading the Cognitive State of Patients for the Clinician. J. Psychiatr. Res. 12:189-198 (1975). As is known, the MMSE is based on a scale of 0-30. An MMSE score of 27 and above is considered normal. An MMSE score of 11 to 26 indicates that the subject has mild to moderate AD-related dementia, and an MMSE score of 0-10 may be characterized as severe AD-related dementia.

The Clinical Dementia Rating (CDR) test is another well-known scoring system used in clinical trials and longitudinal research projects to rate the presence and severity of cognitive problems in subjects with AD and related disorders. The CDR is generated from a semi-structured interview with the patient and a knowledgeable collateral source, such as a spouse or adult child. The CDR is derived using information from the clinical assessment but without reference to psychometric performance, and rates cognitive function in six categories, namely, memory, orientation, judgment and problem solving, community affairs, home and hobbies and personal care. The global CDR is derived by synthesizing ratings in each of the six categories where a CDR score of CDR=0 indicates no dementia, a CDR score of CDR=0.5 signifies uncertain or very mild dementia, and a CDR score of CDR=1, 2, or 3 corresponds to mild, moderate, or severe dementia. Galvin, J. E., et al., The “Portable” CDR: Translating the Clinical Dementia Rating Interview Into a PDA Format, Alzheimer Dis. Assoc. Disord. (2009).

Initial manifestations of the symptoms of AD are typically slow to occur and the existence of very mild dementia (MMSE score greater than 24) can be extremely difficult to detect. For instance, early indications of AD may be nothing more than mild forgetfulness. In this early stage of AD, individuals may have a tendency to forget recent events, activities, and/or the names of familiar people or things. The afflicted individual may not be able to solve simple mathematical problems. As the disease progresses into more moderate stages of AD, symptoms are more easily detected and become serious enough to cause individuals afflicted with AD, or their family members, to seek medical help. More moderate stage symptoms of AD (MMSE score in the range of 11 to 26) typically include the inability to perform simple tasks such as grooming, and problems in speech, understanding, reading, and writing. Severe stage AD patients (MMSE score in the range of 0-10) may have symptoms of anxiousness or aggressiveness. They may wander away from home. Ultimately, and unfortunately, severe stage AD patients may ultimately need complete care and supervision.

In order to provide better care for people who have a predisposition to AD or who have asymptomatic AD, it would be desirable to provide improved methodologies and techniques for prediction, detection, monitoring, treatment, and, generally, diagnosis and prognosis of the disease. It is apparent that early detection of AD would provide opportunities for more proactive and sophisticated care, particularly as advances in treatment of AD are discovered.

In addition, it can also be important to quantitatively distinguish between varying levels of severity of AD so as to design, or tailor, a treatment most efficacious to the known condition of the patient. In other words, treatments for a patient suspected of having AD, or known to have AD, could be designed based on differentiation of asymptomatic AD from mild AD, differentiation of mild AD from moderate AD, and differentiation of moderate AD from severe AD.

Efforts to detect AD in asymptomatic patients at the earliest possible opportunity and to definitively distinguish between varying levels of severity of AD have been made. While good, these efforts are not optimally satisfactory. For example, strategies for detection of saliva-based biomarkers potentially indicative of AD are known. While satisfactory at confirming AD in patients with symptoms of the disease, these saliva-based biomarker strategies are not sufficiently discrete to effectively and accurately identify AD in asymptomatic patients. Moreover, existing salivary biomarker detection strategies are less than optimally effective at repeatably identifying and differentiating varying levels of severity of AD in the patient.

Other approaches to detection of AD in a patient are known but these methodologies have certain drawbacks. Multi-modal strategies are one such example. In a multi-modal strategy, an imaging technique such as Positron Emission Tomography (PET), Computerized Tomography (CT), or Magnetic Resonance Imaging (MRI) is used to detect biomarkers in a patient's cerebrospinal fluid (CSF). However, these multi-modal methods are highly invasive, expensive, and have not been shown to be reliable in terms of sensitivity and specificity to detect AD and different levels of severity of AD with the needed accuracy.

Yet another strategy for identification of AD in a patient involves detection of blood-borne biomarkers. While potentially effective at detecting certain blood-borne biomarkers, removal of blood from a patient is invasive and relatively expensive and, therefore, such assays are not optimally advantageous.

There is a need for improved methods, systems, kits, and other techniques and discoveries for the prediction, detection, monitoring and/or treatment of AD in subjects, which would provide accurate diagnostic information indicative of whether a subject has AD as well as the severity of any such disease, which would provide diagnostic information indicative of AD early in the onset of AD including in subjects who are asymptomatic of AD, which would be non-invasive to the subject, which would be relatively easy to perform without any necessity for use of complex and costly imaging equipment, which could be performed in a wide range of physical settings where one subject or a large population of people could be evaluated for the existence of AD, which can be relatively inexpensive to administer thereby providing an opportunity to serve a greater population of subjects while reducing the overall cost of AD-related care and costs to the healthcare system in general, and which would otherwise represent an advance in the art thereby providing opportunities for improvements in healthcare related to AD.

### SUMMARY

Described herein are methods, systems, kits, and other techniques and discoveries for the prediction, detection, monitoring, treatment, and/or general diagnosis and prognosis of Alzheimer disease (AD) in patients. Aspects of the invention may be implemented to detect, diagnose, and treat patients who are asymptomatic of AD early in the disease process. Other aspects of the invention may be implemented to identify and differentiate the level of severity of AD in a patient such as, for example, identifying mild AD in a subject who is completely asymptomatic of AD, differentiating mild AD from moderate AD, and differentiating moderate AD from severe AD.

The inventions described herein are based on the recognition that certain salivary biomarkers are highly predictive of AD in a subject who may otherwise be asymptomatic of the disease and that detection of such biomarkers as described herein can be used to differentiate between healthy control subjects (i.e., non-cognitive impairment subjects) and patients with cognitive impairment indicative of AD, as well as to differentiate between different levels or categories of severity of AD in the subject. Detection of this biological information in a subject's saliva can, in turn, be used to diagnose, monitor, and treat AD, providing opportunities for earlier and better healthcare outcomes.

It has been found that salivary biomarkers yield particularly reliable results in detection of AD and the severity of the disease. Salivary biomarkers which correlate strongly with AD may be one, or combinations of more than one, of: Insulin-like growth factor binding protein-2 (IGFBP-2), Insulin-like growth factor binding protein-3 (IGFBP-3), Beta-secretase 1 (BACE1), Reduced glutathione (GSH), TNF-related apoptosis-including ligand (TRAIL), Interleukin 6 (IL-6), Chitinase-3-like protein 1 (YKL-40), ICAM-1, Vascular cell adhesion protein 1 (VCAM-1), Neurofilament protein L (NfL), Alpha-1 antitrypsin (A1AT), Transthyretin (TTR), Neurogranin, and Human heart fatty acid binding protein (hFABP).

Besides yielding highly accurate biomarker information indicative of AD, saliva as a biomarker source has other unique and compelling advantages. Advantages include noninvasiveness of collection, ease of analysis, fast and easily understood results, and low cost of administration providing opportunities for saliva-based methods, systems, and kits that can be implemented to detect AD for one or many subjects on a widespread, low-cost basis. The ability to implement saliva-based detection of AD provides opportunities to screen large populations of aging people for the presence or absence of AD.

In embodiments, a method of detecting biomarkers indicative of the AD in a human subject is provided. The method may include obtaining a saliva sample from the subject and detecting whether one or more biomarkers indicative of AD is present in the saliva sample. Examples of salivary biomarkers which may be detected include the aforementioned IGFBP-2, IGFBP-3, BACE1, GSH, TRAIL, IL-6, YKL-40, ICAM-1, VCAM-1, NfL, A1AT, TTR, Neurogranin, and hFABP separately, or in combinations.

Many types of assays may be implemented to detect the biomarker(s) of interest, examples of which include lateral flow immunochromatographic assays (LFA), and enzyme-linked immunosorbent assays (ELISA). In examples of these types of assays, agents, such as antibodies with affinity for a type of biomarker, may be contacted by the saliva sample and bind with a specific one of the biomarkers. Such agent or antibodies may be secured to a solid support thus immobilizing the bound biomarkers. Detecting of the binding between the agent and the specific biomarkers may yield a result positive for AD. Based on the extent of detected binding, levels of severity of AD can be ascertained. The detection may further include detecting the lack of, and/or insufficiency of, binding thus yielding a negative result indicative of a healthy subject who is free of AD. In embodiments, detecting can be accomplished by binding of labeled antibodies to immobilized salivary biomarkers providing a visible indication, such as a color change.

In embodiments, the label provided may be measurable indicative of a proportional reaction based on the level of biomarker present in the saliva sample. If desired, a measurement device may be implemented to detect and utilize the label to provide a qualitative, semi-quantitative, or quantitative measure of the one or more salivary biomarker indicative of whether the subject is afflicted with AD or the severity of AD in the subject.

In certain embodiments, the agent or antibodies may provide a visible indication when the at least one biomarker in the saliva sample meets or exceeds a reference value or amount. The reference value or amount may be derived from healthy subjects unaffected by AD so that a visible indication is evidence that the subject is likely to have AD and should seek medical assistance.

Methods may include detecting combinations of salivary biomarkers which are highly predictive of AD and detection of such combinations provides information indicative of AD in a subject. In embodiments, combinations may include BACE1 and NfL, NfL and IGFBP-2, BACE1 and IGFBP-2, NfL and hFABP, NfL and GSH, NfL and IL-6, BACE1 and hFABP, BACE1 and GSH, BACE1 and IL-6, IGFBP-2 and hFABP, IGFBP-2 and GSH, IGFBP-2 and IL-6, IGFBP-2 and IGFBP-3, and IGFBP-2 and Neurogranin and the agents or antibodies may have affinity for such combinations.

Detection of the combination of IGFBP-2, IGFBP-2, and BACE1 or the combination of IGFBP-2, Neurogranin, and GSH are highly indicative of AD in a subject and such combination can be identified in an assay with a desirably small group of three different agents or antibodies. Further accuracy can be provided by additional implementation of agents or antibodies capable of binding with additional ones of IGFBP-2, IGFBP-3, BACE1, GSH, TRAIL, IL-6, YKL-40, ICAM-1, VCAM-1, NfL, A1AT, TTR, Neurogranin, and hFABP. Methods and assays to detect yet other combinations may be implemented as described herein.

The invention may be implemented in the form of a kit and/or a system. In embodiments, a kit for detecting salivary biomarkers indicative of AD in a subject may include an assay. The assay may have a solid support on which one or a plurality of agents have been affixed, directly or indirectly, and which bind to one or more biomarker in a saliva sample obtained from the subject. The solid support could be provided as part of an LFA, or an ELISA, or another type of assay. The agent or agents may have an affinity for one or more of the aforementioned IGFBP-2, IGFBP-3, BACE1, GSH, TRAIL, IL-6, YKL-40, ICAM-1, VCAM-1, NfL, A1AT, TTR, Neurogranin, and hFABP biomarkers and each agent may bind to a different single type of biomarker. Kits tailored to detect biomarker combinations such as those previously described may be implemented. In embodiments, the agent or agents may be antibodies. Additional labeled antibodies with an affinity for specific ones of the biomarkers may be utilized to enable formation of a visible complex if one or more of the biomarkers is present in the saliva sample.

Detection of the visible complex, such as by a color change, yields a result positive for AD and the extent of detected binding enables levels of severity of AD to be determined. Detecting that a visible complex has not formed or has formed insufficiently provides a result indicative that the subject is healthy.

In embodiments, a system for detecting biomarkers indicative of AD in a saliva sample may include an assay with at least one binding agent specific to one or more biomarker according to the previously-described embodiments, a measurable label that indicates a proportional reaction based on the amount of biomarker present in the saliva sample, and a measurement device operable to utilize the label to provide a qualitative and/or quantitative measure of the one or more biomarker indicative of whether the subject is afflicted with AD. Measurement devices which may be implemented to detect the amount of the label may include optical-type readers.

The invention may be implemented as part of a treatment program to ascertain the effectiveness of pharmaceutical agents in treating or lessening the symptoms of AD in a subject. Changes in the aforementioned one or more of IGFBP-2, IGFBP-3, BACE1, GSH, TRAIL, IL-6, YKL-40, ICAM-1, VCAM-1, NfL, A1AT, TTR, Neurogranin, and hFABP biomarkers responsive to a pharmaceutical agent may be utilized to determine efficacy of the treatment.

The invention may be implemented as part of a method for detecting whether a subject who is asymptomatic of AD is actually afflicted with AD.

These and other embodiments and specific and possible advantages will become evident with reference to the following description.

### DETAILED DESCRIPTION

The present invention relates to improvements in the prediction, detection, monitoring, treatment, and general diagnosis and prognosis of the neurological disorder referred to as Alzheimer's disease (AD). Methods, systems, and kits according to the invention may be implemented by means of saliva harvested from a subject and include assaying of a saliva sample for the presence of one, more than one, or multiple different biomarker combinations which correlate strongly with the existence, severity, and progression of AD in the subject. The subject is most preferably a human subject. Certain embodiments may be implemented ex vivo in that they can occur apart from the subject.

The correlation of the salivary biomarkers with AD is strong in symptomatic subjects and, importantly, in asymptomatic subjects, providing a powerful tool by which to identify asymptomatic people and to provide opportunities for early treatment of AD. Biomarker information may be further used to determine or estimate the effectiveness of a particular treatment in limiting and/or reversing progression of AD in a subject. The ability to implement the invention to obtain the aforementioned types of information by means of assaying saliva provides important opportunities for the accurate, rapid, non-invasive, and inexpensive testing of one subject, or many subjects, for affliction with AD.

Methods, systems, and kits according to the invention may be implemented in any location including at a hospital, a clinic, a laboratory, as well as in the “field” at a needed location apart from any medical or laboratory facility. For example, the invention may be implemented at a physician's office, a satellite clinic, an outpatient facility, and other non-traditional testing facilities where, for example, large populations of people could be evaluated for the existence of AD.

As described herein, it has been found that certain biomarkers and combinations of biomarkers, if present in saliva, have characteristics useful in the prediction, detection, monitoring, treatment, and general diagnosis and prognosis of AD. In embodiments, the invention may be implemented by means of one, two, or combinations of more than two, of the biomarkers of the group including: Insulin-like growth factor binding protein 2 (IGFBP-2), Insulin-like growth factor binding protein 3 (IGFBP-3), Beta-secretase 1 (BACE1), Reduced glutathione (GSH), TNF-related apoptosis-including ligand (TRAIL), Interleukin 6 (IL-6), Chitinase-3-like protein 1 (CHI3L1), also known as and referred to herein as YKL-40, ICAM-1, Vascular cell adhesion protein 1 (VCAM-1), Neurofilainent protein L (NfL), Alpha-1 antitrypsin (A1AT), Transthyretin (TTR), Neurogranin, and Human heart fatty acid binding protein (hFABP).

These biomarkers, or biomarker combinations, have been found to correlate strongly with the existence, severity, and progression of AD in a human subject. It is envisioned that other biomarkers having similar characteristics to those listed above, such as proteins, peptides, and genetic and transcriptomic organic and inorganic biomarkers in saliva may also have utility in detection of AD in a subject as well as the severity of AD.

Methods, systems, and kits for assaying saliva samples according to the invention may be implemented in many different ways according to the needs of the medical professional, technician, care giver, and/or the diseased subject. Modes of implementation may include, for example, assays such as lateral flow immunochromatographic assays (LFA), enzyme-linked immunosorbent assays (ELISA), a ready-to-use assay device, a “lab-on-a-chip”, or even as a biosensor accessory for use with a mobile device such as an iOS-based iPhone or iPad or with an Android-based mobile device. Salivary biomarkers may be qualitatively or quantitatively measured using these and other assaying strategies for the prediction, detection, monitoring, treatment, and general diagnosis and prognosis of AD.

As described herein, biomarkers of the types described herein are particularly useful in detection of AD in patients who are asymptomatic of AD, or mildly symptomatic of AD, at a point in the AD disease process earlier than can be diagnosed by means of existent techniques. The biomarkers of the invention provide surprisingly accurate results in detecting the severity of the AD in a patient correlating highly with the MMSE scale, including mild, moderate, and severe AD.

Before describing examples of the invention, definitions of certain terms used in this patent application are provided.

### Definitions

As used in this document, the singular forms “a”, “an”, and “the” include plural references unless the context clearly dictates otherwise.

As used herein, the terms “Alzheimer's patient or subject” and “AD patient or subject” each refers to an individual who has been diagnosed with AD. The individual may be diagnosed with AD by any means generally used by those of skill in the art. For example, he or she may be diagnosed through a Mini-Mental State Examination (MMSE) or any other recognized method or combinations of methods, such as the Clinical Dementia Rating (CDR). As previously described, an MMSE score of 27 and above is considered normal. An MMSE score of 11 to 26 indicates that the subject has mild to moderate AD-related dementia, and an MMSE score of 0 to 10 may be characterized as severe AD-related dementia.

“Assaying” means or refers to the analysis of a saliva sample to determine the presence of one or more salivary components, referred to herein as biomarkers. The assaying may be performed using many different processes in accordance with the subject matter disclosed herein. Non-limiting types of assays which may be implemented according to the invention include the aforementioned LFA and ELISA types of assays.

A “biomarker”, also known as a biological marker, means or refers to a measurable indicator of a biological state or condition. As described herein, examples of biomarkers which have been determined to be indicative of AD in a subject may include one or more of: IGFBP-2, IGFBP-3, BACE1, GSH, TRAIL, IL-6, YKL-40, ICAM-1, VCAM-1, NfL, A1AT, TTR, Neurogranin, and/or hFABP.

A “biomarker panel” defines a set of biomarkers used alone, in combination, or in sub-combinations for the prediction, detection, monitoring, treatment, and general diagnosis and prognosis of a disease or condition based on detection values for the set of biomarkers.

As used herein, the terms “comprising”, “including”, “containing”, “consisting of”, and “characterized by” are interchangeable, inclusive and open-ended and do not exclude additional biomarkers, methods or procedural steps.

A “normal” patient or subject or sample from a normal patient or subject as used herein for quantitative and qualitative data refers to an individual who has or would be assessed by a physician as not having AD, and has an MMSE score or would achieve a MMSE score in the range of 27-30 and above. A normal patient or subject is generally age-matched within a range of 5 to 10 years, including but not limited to an individual that is age-matched, with the individual to be assessed.

An “asymptomatic” case of AD means or refers to a patient or subject who has a confirmed case of the disease but who lacks any relevant clinical symptoms of AD.

“MCIAD” means or refers to mild cognitive impairment with probable early AD. MCIAD subjects refers to people who are afflicted with AD but in a very mild form with minimal cognitive impairment. MCIAD subjects are people with a typical MMSE score range of about 26.5 to about 26.8 or a CDR score of 0.5. MCIAD subjects typically have no outward symptoms of AD and may be considered asymptomatic for this reason.

A “mild” case of AD means or refers to a subject with mild symptoms which cannot be classified as severe. An MMSE score of 11 to 26 or CDR score of 1 are indications that the subject has mild to moderate AD-related dementia.

A “severe” case of AD means or refers to a subject showing any of the following severe symptoms associated with severe AD: progressive memory loss, cognitive deficits, and dementia resulting in impaired functions in daily living and behavioral symptoms. Impairment of cognitive functions results in difficulties in coping with both complex and simple repetitive activities such as daily planning, working, managing finances, preparing food, keeping order, socializing, or pursuing interests. An MMSE score of 0 to 10 may be characterized as severe AD-related dementia.

“Detecting”, “measuring”, or “taking a measurement” define a qualitative or quantitative determination of the amount, or level, or concentration of a biomarker in the sample, including the absence of the biomarker.

A “measurement device” means or refers to any device operable to provide a qualitative and/or quantitative level of one or more biomarker in a sample.

The words: “evaluate”, “differentiate”, “determinate”, “discriminate” and “establish” are used for diagnosis, prognosis, and monitoring and these words are interchangeable.

“Ex vivo” means or refers to experimentation or measurements done in an environment external to a subject.

As used herein, a “reference value” of a biomarker may be any of a relative value, an absolute value, a range of values, a value that has an upper and/or lower limit, an average value, a median value, a mean value, a value as compared to a control or baseline value, or a combination thereof. A reference value may also be articulated as a level or an amount, or a concentration.

“Subject” or “individual” or “patient” means or refers to a human being. In certain embodiments, it is possible that a subject, individual, or patient may also refer to a non-human mammal, such as a primate, or a murine organism.

As used herein, the term “treatment” means or refers to the alleviation, amelioration, and/or stabilization of symptoms, as well as delay in progression of symptoms of a particular disorder. For example, “treatment” of AD includes any one or more of: elimination of one or more symptom of AD, reduction of one or more symptom of AD, stabilization of one or more symptom of AD (e.g., failure to progress to more advanced stages of AD), and delay in progression (i.e., worsening) of one or more symptom of AD.

Insulin-like growth factor binding protein 2 (IGFBP-2) is a protein that in humans is encoded by the IGFBP2 gene.

Insulin-like growth factor binding protein 3 (IGFBP-3) is the main carrier of somatomedin C (also known as insulin-like growth factor-1, or IGF-1) in the body which is encoded by the IGFBP3 gene in humans.

Beta-secretase 1 (BACE1) is an enzyme that in humans is encoded by the BACE1 gene. BACE1 is an aspartic acid protease important in the formation of myelin sheaths in peripheral nerve cells.

Reduced glutathione (GSH), is an antioxidant present in almost every cell in the body which is playing a role in the detoxification of drugs and xenobiotics. Also, GSH acts as a hydrogen donor in the detoxification of hydrogen peroxide. Burk, R. F., Glutathione-dependent protection by rat liver microsomal protein against lipid peroxidation. Biochim Biophys. Acta. 757(1):21-28 (1983).

TNF-related apoptosis-including ligand (TRAIL) is a protein functioning as a ligand that induces the process of cell death called apoptosis. There is expression of TRAIL in various cells such as dendritic cells, T cells, natural killer cells, and monocytes indicative that TRAIL participates in modulating the body's immune function and that TRAIL has an effect on host defense and immune homeostasis. Finnberg N. et al., TRAIL-R Deficiency in Mice Promotes Susceptibility to Chronic Inflammation and Tumorigenesis, J. Clin. Invest. 118(1):111-123 (2008).

Interleukin 6 (IL-6) is an interleukin that acts as a pro-inflammatory cytokine. In humans, it is encoded by the IL6 gene.

Chitinase-3-like protein 1 (CHI3L1), also known as and referred to herein as YKL-40, is a secreted glycoprotein encoded by the CHI3L1 gene.

ICAM-1 is a protein that in humans is encoded by the ICAM1 gene.

Vascular cell adhesion protein 1 (VCAM-1) is a protein that in humans is encoded by the VCAM1 gene. VCAM-1 functions as a cell adhesion molecule.

Neurofilament, protein L (NfL) is a low molecular weight neurofilament protein.

Alpha-1 antitrypsin (A1AT) is a protein belonging to the serpin superfamily. It is encoded in humans by the SERPINA1 gene and is a protease inhibitor.

Transthyretin (TTR) is a transport protein in the serum and cerebrospinal fluid that carries the thyroid hormone thyroxine (T4) and retinol-binding protein bound to retinol.

Neurogranin is a calmodulin-binding protein expressed primarily in the brain, particularly in dendritic spines, and participation in the protein kinase C signaling pathway.

Human heart fatty acid binding protein (hFABP) is a protein that is encoded by the FABP3 gene.

As used herein, the term “about” when used in connection with a numerical value means or refers to the value+/−10% indicative that variance from the value is permissible in accordance with the invention.

The terminology used herein is for the purpose of describing specific embodiments only and is not intended to be limiting of the invention. Unless defined otherwise, all technical and scientific terms used herein have the same meanings as commonly understood by one of normative skill in the art.

The inventors have discovered that certain biomarkers, sets or groups of biomarkers, and methods of detection of biomarkers present in the saliva of subjects are changed in those subjects afflicted with Alzheimer's disease (AD). Accordingly, these biomarkers, and sets or groups of biomarkers, may be used to predict AD with respect to a subject, detect AD in a subject, monitor and quantifiably measure progression of AD in a subject, treat a subject afflicted with AD, and generally develop and provide a diagnosis and prognosis of a subject for AD. The invention presents methods, systems, kits, and technology for the prediction of AD with respect to a subject, detection of AD in a subject, monitoring and measuring of progression of AD in a subject, treating a subject afflicted with AD, and generally providing a diagnosis and prognosis of a subject for AD by semi-quantitatively and quantitatively measuring the concentration of each of a series of AD diagnostic biomarkers in saliva samples.

Furthermore, this invention describes a means for quantification of single or multiple biomarkers, which, when measured singly or in combination, are strong indicators for diagnosis of AD, the prognosis of AD, early detection of AD, treatment of AD, and risk of AD in the individual. Furthermore, this invention identifies combinations of biomarkers that, when used in prediction, detection, monitoring, treatment, management, and general prognosis and diagnosis of AD, are highly sensitive and specific for AD.

The methods described herein provide for non-invasive, painless, and stress-free assessment and classification of AD risk factors as indicators for neurological disease using saliva as a diagnostic fluid. The saliva from the subject of interest may be used in conjunction with immunological assay detection technologies to quantify the levels of the various biomarkers in the subject's saliva specimen(s).

Iterations of the invention are highly effective in detection of AD in human subjects who are asymptomatic of AD in a manner which correlates highly with MMSE and CDR scoring of AD. These iterations can be used as a diagnostic tool separate from, or in combination with, MMSE and CDR evaluations to detect AD in the otherwise asymptomatic subjects. Subjects with MMSE scores of 27 and above appear by all outward mannerisms to be normal and yet such subjects may actually be in the earliest stages of AD. Another group of asymptomatic subjects are those known by the acronym MCIAD. These MMSE and CDR scores position MCIAD subjects in a grouping between normal healthy subjects with AD affliction and subjects with mild to moderate AD as defined herein.

Early detection of AD in subjects with no symptoms of AD, or with just mild symptoms of AD is of importance. Early detection of AD provides an opportunity for early medical intervention and treatment and therefore provides an opportunity for improved healthcare outcomes.

Biomarkers and biomarker combinations comprising one, two, or combinations of more than two of the biomarkers IGFBP-2, IGFBP-3, BACE1, GSH, TRAIL, IL-6, YKL-40, ICAM-1, VCAM-1, NfL, A1AT, TTR, Neurogranin, and hFABP have certain characteristics which make them effective in detecting, diagnosing, treating, managing, and generally providing a diagnosis and prognosis of AD. Without wishing to be bound by any particular theory, it is believed that biomarkers of the present invention provide for surprisingly more accurate detection of AD for several reasons including at least the following non-limiting reasons:

First, it has been discovered that the aforementioned biomarkers and biomarker combinations are highly expressed in the saliva of subjects afflicted with AD as compared with healthy subjects who are free of the disease, readily providing comparative information useful in detecting, diagnosing, treating, managing, and providing a diagnosis and prognosis of AD.

Second, biomarkers as described herein are expressed early in the AD disease process. Early expression in the AD disease process provides opportunities for early identification of AD and earlier treatment of the subject for AD. For example, IGFBP-2 and IGFBP-3 are present as precursors to IGF-I and IGF-II. IGFBP-2 and IGFBP-3 can potentiate the action of IGF-I and IGF-II. IGFBP-2 and IGFBP-3, however, act independently of IGF-I and IGF-II. Bach, L. A., What Happened to the IGF Binding Proteins? Endocrinology 159: 570-578 (2018). Moreover, IGF I and IGF II activity are modulated and controlled by IGFBP-2.

Third, biomarkers of the types described herein are relatively easy to handle and use, again improving confidence in the diagnostic conclusions that can be drawn from the information. For example, existent biomarkers require special processing including required use of stabilizers and also centrifugation to remove impurities from the saliva before the biomarkers can be identified and analyzed. In contrast, biomarkers of the types described herein may be analyzed without stabilized saliva and with our without centrifugation. For example and as illustrated in Example 1 below, the biomarkers utilized in that example are evaluated without any necessity for use of stabilizers thereby illustrating the stability of the biomarkers.

Fourth, biomarkers of the types described herein are stable for a time duration of 20-30 days, and an even greater number of days. The stability of the biomarkers of the invention can be appreciated by comparison of, for example, NfL and a protein such as TNF alpha. Both NfL and TNF alpha are indicators of an inflammatory pathway related to AD. However, the stability of NfL is much better than that of TNF alpha. One measure of biomarker stability is biomarker concentration after a period of days. The biomarker NfL remains intact and has a consistent concentration as compared to TNF alpha which has a significantly reduced concentration over a 20 day period at 37° C.

Fifth, biomarkers of the types described herein are found in relatively elevated concentrations in saliva of people afflicted with AD as compared with existent biomarkers. For example, the known biomarkers Aβ-40 and Aβ-42 are typically present in saliva in the range of 2.8 to about 25.85 pg/ml and 4.6 to about 19.6 pg/ml respectively. In contrast and as will be demonstrated herein, BACE1, a product of the amyloid pathway, is typically present in the saliva of a person afflicted with AD in an amount of about 800 pg/ml to about 1270 pg/ml making BACE1 relatively easier to detect in an AD subject. Existence of biomarkers as disclosed herein at relatively greater concentrations in the saliva of people afflicted with AD provides an improved opportunity to detect the biomarkers and reach accurate conclusions regarding a diagnosis of whether the subject is afflicted with AD.

The present discoveries are not limited to human subjects. Any mammal thought to suffer from AD may benefit from the present invention.

The present inventors have recognized that human saliva is a uniquely valuable source of biological information enabling repeatable detection, diagnosis, monitoring, and treatment of AD in a subject. Saliva refers to the watery liquid secreted into the mouth by glands. Saliva serves as an aid in digestion and provides lubrication for chewing and swallowing. Saliva is such a valuable bodily fluid because it contains the biomarkers IGFBP-2, IGFBP-3, BACE1, GSH, TRAIL, IL-6, YKL-40, ICAM-1, VCAM-1, NfL, A1AT, TTR, Neurogranin, and hFABP which are indicative of AD. Saliva has been found to have greater concentrations of the aforementioned biomarkers than bodily fluids such as blood, urine, and tears providing an opportunity for more accurate results.

Saliva samples can be easily harvested, or collected, from human subjects which is another reason that saliva is an ideal source of biomarker information. Collection of saliva is non-invasive, stress-free to the subject, can be conducted in real time, and can be done with a person having minimal technical training. Bodily fluids such as blood, urine, tears, lack these advantages.

Saliva sample volumetric sizes of from about 1 ml to about 5 ml are sufficient for assaying according to aspects of the invention. Saliva harvesting may be stimulated or unstimulated. Stimulated saliva production can be achieved by insertion of a wand-like oral appliance into a subject's mouth followed by chewing or sucking on the appliance. Excess saliva is deposited into a tube, a vial, or another container. Unstimulated saliva production may involve relaxed drooling from the subject's lower lip into a tube, a vial, or another container. A 2% sodium azide solution may be added to each saliva sample to prevent microbial decomposition of the saliva.

Unstimulated saliva production may require about 10 to about 15 minutes to collect a 1 ml to about 5 ml of saliva sample depending on the subject. Typically, the subject is asked to rinse orally with water ten to fifteen minutes prior to collection of unstimulated saliva samples. Following collection, the saliva samples may be centrifuged at, for example, 1800 rpm for 5 minutes to remove debris. The centrifuged saliva samples may then be frozen or placed in an ice bed to await further analysis.

The present invention enables assaying of a saliva sample harvested or collected from a subject to predict AD, detect AD, monitor AD, treat AD, and to generally provide a diagnosis and prognosis of AD in a subject.

In general, a method according to the invention comprises the steps of (a) obtaining a saliva sample from a subject and (b) detecting whether one or more biomarkers selected from the group consisting of IGFBP-2, IGFBP-3, BACE1, GSH, TRAIL, (IL-6), YKL-40, ICAM-1, (VCAM-1), NfL, A1AT, TTR, Neurogranin, and hFABP, is or are present in the saliva sample by contacting the saliva sample with an agent capable of binding with a specific one of the biomarkers and detecting the binding between the agent and the biomarker specific thereto.

Qualitative or quantitative measurement of the level or amount of the detected biomarkers may be conducted ex vivo of the subject utilizing the subject's saliva sample. In embodiments, the measured amount of the biomarker(s) can be compared to a reference value or amount (e.g. a concentration) of the biomarker(s) derived from subjects who are healthy (i.e., a control) and who are not afflicted with AD. If the measured amount of biomarker exceeds the reference value or amount, that outcome would be indicative that the subject is afflicted with AD, whereas amounts below the reference value or amount would be indicative that the subject is not afflicted with AD. This information could be particularly valuable in determining whether an asymptomatic subject is afflicted with AD.

In embodiments, a qualitative measurement of the detected biomarker may include an assay in which a determination is made regarding whether the amount of biomarker in the saliva sample exceeds a reference value or amount or level, also referred to herein as a “cutoff” level. If the detected biomarker exceeds the cutoff level, that could trigger identification of a test line or lines on a lateral flow strip assay or change the color of a test pad in some visually-observable manner, thus providing a binary yes/no result indicative of AD or, much preferably, lack of AD.

In other embodiments, a quantitative measurement of detected biomarker may be conducted in which the strength of a color, fluorescence, or some other indicator can be used to quantify the amount of biomarker in the saliva sample. Quantitative measurement of detected biomarker may be used to determine whether the amount of biomarker exceeds the reference value or amount or level (i.e., the “cutoff” level) and may be useful to quantitatively determine the progression and severity of the infection (e.g., between asymptomatic, moderate, and severe states of infection). Quantitative changes in the amount or level or concentration of biomarkers in a subject's saliva when evaluated over a time period (e.g., hours, days, months, etc.) may further be used to determine or estimate the effectiveness of a particular treatment in limiting and/or reversing progression of a viral infection or other disorder.

Methods according to the invention may be implemented with different types of assays. Examples of assays which can be implemented for purposes of detecting salivary biomarkers indicative of AD in a subject may include, without limitation, (1) lateral flow immunochromatographic assays (LFA), (2) enzyme-linked immunosorbent assays (ELISA), (3) enzyme-linked fluorescence polarization immunoassays (FPIA), (4) homogeneous immunoassays, (5) quantitative point-of-care tests using determination of chemiluminescence, fluorescence, magnetic particles, and latex agglutination, (6) gel electrophoresis, (7) gas chromatograph-mass spectrometry (GC-MS), (8) separation immunoassays, (9) heterogeneous immunoassays, (10) homogenous immunoassays, (11) latex agglutination, (12) western blot analysis, (13) biosensor technology using fluorescence, chemiluminescence, magnetic bead-based technologies and others, as well as (14) alternate newer or upcoming technologies including nanotechnology and assays which will be developed in the future. Biosensors and lab-on-a-chip methodologies may be implemented thereby introducing the possibility of a test for AD capable of being performed in a clinic, a physician's office, or in the form of a home-based AD test. Furthermore, tests may be based upon one of a series of available rapid testing technologies and may, or may not, need a reading device (e.g., a hand-held reading device) to read and quantify the levels of the various biomarkers in saliva specimens collected from one or more subject. Integration of the salivary biomarker technology described herein with different point-of-care and biosensor platform devices, or other similar technologies, will enable immediate results to be obtained through diagnosis at the point-of-care in a cost effective manner without sophisticated equipment or instrumentation. ELISA is a preferred technique because of its ease of use.

The present invention provides methods, kits, and systems for prediction, detection, monitoring, treatment, and general prognosis and diagnosis of Alzheimer's disease (AD), comprising comparison of a measured level of a number of different AD biomarkers in a saliva sample from an individual seeking a diagnosis for AD compared to a reference level for each biomarker, wherein the different AD biomarkers are one, or more than one, of IGFBP-2, IGFBP-3, BACE1, GSH, TRAIL, IL-6, YKL-40, ICAM-1, VCAM-1, NfL A1AT, TTR, Neurogranin, and hFABP.

In some embodiments, methods, kits, and systems may comprise comparing the measured value of the biomarker or biomarkers to a reference value for each such biomarker and may also consist of calculating the number of fold differences between the measured value and the reference value. In other embodiments, methods, kits, and systems may comprise comparing the fold difference for each AD biomarker measured with a minimum fold difference value.

In another embodiment, the statistically significant difference is measured in terms of a p-value, where the p-value ranges from 0 to 0.05, while in other embodiments, parameters for the statistically significant difference comprise one or more of a correlation of greater than 70% (r=0.7 to r=0.99), a p-value of between 0 and 0.05, a fold change in levels of greater than 20%, and a “d” score (a measure of the decrease or increase in specific levels of biomarkers in AD patients). In some embodiments, the measured levels are normalized against values from normal healthy individuals. In certain embodiments, the reference levels are obtained from measured values of the different biomarkers from samples in the saliva of human individuals without AD. In some embodiments, the reference levels are obtained from measured values of the different biomarkers from samples in the saliva of human individuals with AD. In some embodiments, the methods, kits, systems and technology include comparing the measured level of salivary biomarkers diagnostic of AD to two reference levels for each biomarker. In some embodiments, the two reference levels for each biomarker comprise: (a) a reference level obtained from measured values of different biomarkers from samples in saliva of human individuals without AD; and (b) a reference level obtained from measured values of biomarkers from samples in the saliva of human individuals with AD.

In some embodiments, the group of individuals without AD is a control population selected from an age-matched population, a degenerative control population, a non-AD neurodegenerative control population, a healthy age-matched control population, or a mixed population thereof. This method is a non-invasive, painless classification of the neurological AD using saliva as a diagnostic fluid, which, when used in conjunction with a point-of-care device, introduces the possibility of a home-based dementia assessment test. Use of the technology may also be applied to a multitude of other technologies available for diagnosis under laboratory and field conditions, with and without instrumentation. Such methods include, without limitation, LFA, ELISA, homogeneous immunoassays, mass spectrometry, latex agglutination, FPIA, chemiluminescence immunoassays, biosensor technology, microsphere-based capture-sandwich immunoassay devices, multiplexed immunoassay devices, and multiplexed sandwich immunoassay devices, among others.

In certain embodiments, the group of individuals without AD is a control population selected from an age-matched population, a degenerative control population, a non-AD neurodegenerative control population, a healthy age-matched control population, or a mixed population thereof. In some embodiments, all group individuals are a minimum of 40 years of age and a maximum of 90 years of age. In some embodiments, said individual is at least 40, 45, 55, 60, 65, 70, 75, 80, 85 or 90 years of age. In another aspect of the invention, laboratory based tests are used to measure the values and/or reference levels. Provided herein are methods for obtaining comparative values for measured levels relative to reference levels in biological fluid samples, particularly saliva. In any of the above embodiments, the comparison of the measured value and the reference value includes calculating a fold difference between the measured value and the reference value. In some embodiments, the measured value is obtained by quantifying the level of various AD diagnostic biomarkers in available patient samples, while in other embodiments the measured value is obtained from data from collection of samples carried out at five independent clinics.

Embodiments of the present invention provide methods of diagnosing AD including the steps of: (a) preparing magnetic particles having primary capture antibodies specifically bonded with IGFBP-2, IGFBP-3, BACE1, GSH, TRAIL, IL-6, YKL-40, ICAM-1, VCAM-1, NfL, A1AT, TTR, Neurogranin, and/or hFABP adsorbed thereon, (b) introducing saliva containing IGFBP-2, IGFBP-3, BACE1, GSH, TRAIL and IL-6, YKL-40, ICAM-1, VCAM-1, NfL, A1AT, TTR, Neurogranin, and/or hFABP into the magnetic particles to bond the beta-amyloid contained in the saliva with the primary capture antibodies, (c) bonding secondary capture antibodies labeled with fluorescent substances to the magnetic particles bonded with beta-amyloid to form a complex, (d) disposing the complex in a channel region of a photoelectric conversion device in which photoelectric current is changed according to an amount of incident light, and (e) using a measurement device to measure photoelectric current changed by light excited from the complex to quantify a concentration of the biomarkers or biomarker combinations, namely, the IGFBP-2, IGFBP-3, BACE1, GSH, TRAIL, IL-6, YKL-40, ICAM-1, VCAM-1, NfL, A1AT, TTR, Neurogranin, and/or hFABP contained in the saliva.

In some embodiments, the photoelectric conversion device may include an optical filter layer transmitting a wavelength of excitation light excited from the fluorescent substances.

In other embodiments, the photoelectric conversion device might include a semiconductor substrate, an insulation layer on the semiconductor substrate, a channel pattern on the insulation layer, and spaced apart interconnection electrodes disposed on the channel pattern.

In still other embodiments, the optical filter layer may be disposed on the channel pattern.

In other embodiments, the fluorescent substances might be formed of a material emitting light having a wavelength band of 650 nm to 850 nm by excitation light having a wavelength ranging from 350 nm to 550 nm.

In other embodiments of the present invention, a method of, and system for, diagnosing AD may include the steps of: (a) preparing comparative samples based on the concentration of biomarkers present in healthy subjects, namely, concentrations of IGFBP-2, IGFBP-3, BACE1, GSH, TRAIL, IL-6, YKL-40, ICAM-1, VCAM-1, NfL, A1AT, TTR, Neurogranin, and/or hFABP, (b) preparing magnetic particle samples having IGFBP-2, IGFBP-3, BACE1, GSH, TRAIL, IL-6, YKL-40, ICAM-1, VCAM-1, NfL, A1AT, TTR, Neurogranin, and/or hFABP contained in each comparative sample combined with multiprotein, (c) measuring changes in photoelectric current from the magnetic particle samples by using a measuring device including an optical field effect transistor, in which photoelectric current is changed according to an amount of light, to generate reference data from the healthy subjects, (d) introducing saliva from a subject suspected of being afflicted with AD and containing one or more of the biomarkers IGFBP-2, IGFBP-3, BACE1, GSH, TRAIL, IL-6, YKL-40, ICAM-1, VCAM-1, NfL, A1AT, TTR, Neurogranin, and/or hFABP to magnetic particles bonded with the multiprotein, (e) measuring changes in photoelectric current from the magnetic particles by using the measuring device and optical field effect transistor to generate measurement data for the subject suspected of being afflicted with AD, and (f) comparing the reference data and the measurement data to diagnose the presence of AD.

Measurement devices may be selected to determine the concentration of biomarker present in the saliva samples based on the type of assay being implemented. Qualitative and/or quantitative measurement may be implemented. For example, a measurement device may comprise an RDS 2500 from Detekt Biomedical LLC for analysis of lateral flow and dry chemistry test strips, an AppDx Smartphone lateral flow test strip reader from Abingdon Health, the Quanterix brand SR-X™ biomarker detection system, and ELISA kits from R&D Systems, Thermo Fisher Scientific, and Arbor Assays. Such measurement devices are capable of detecting and measuring biomarkers captured on a solid substrate thereby providing a quantifiable value indicative of the concentration of the biomarker or biomarkers present in the saliva sample.

Embodiments of the invention may be implemented in the form of a diagnostic kit purposed to assay a saliva sample and to detect one or more biomarkers indicative of AD in the saliva sample. Non-limiting types of assays which may be implemented in a kit according to the invention include LFA and ELISA types of immunoassays of the types described herein. Any of the assays referenced herein may be implemented in the form of a kit.

Advantageously, embodiments of such a kit are capable of being provided in the form of a “ready-to-use” assay which is simple to use, rapid, and produces an easily understood result. Such a kit could be implemented at any location including at a physician's office, a hospital, a clinic, or a laboratory. Such a kit could further be utilized away from these types of institutional settings at a needed location (i.e., in the “field”). For example, a kit according to the invention could be used at a home, a school, a business, a testing node, a point-of-care, or even at public or private events for purposes of screening groups of people for the presence of AD. A “ready-to-use” assay could be portable, lightweight, inexpensive, and easy to use and therefore be capable of widespread use to potentially screen large groups of people.

In general, a diagnostic kit may comprise an assay which receives a saliva sample from a subject. The assay can then detect the presence and quantity of one or more of IGFBP-2, IGFBP-3, BACE1, GSH, TRAIL, IL-6, YKL-40, ICAM-1, VCAM-1, NfL, A1AT, TTR, Neurogranin, and/or hFABP in the sample. In particular, this may be achieved by contacting the saliva sample from a subject with a binding agent or agents that specifically recognize(s) an epitope of one or more of the biomarkers IGFBP-2, IGFBP-3, BACE1, GSH, TRAIL, IL-6, YKL-40, ICAM-1, VCAM-1, NfL, A1AT, TTR, Neurogranin, and/or hFABP.

In one embodiment, the binding agent is an antibody formed in a rabbit or is an immunoglobulin domain thereof. In another embodiment of a kit, the binding agent is an antibody or an antigen binding part thereof comprising an immunoglobulin domain known in the art, or another specific binding agent known in the art such as a ligand, receptor, chemical ligand, aptamer, etc.

The binding agent binds to the IGFBP-2, IGFBP-3, BACE1, GSH, TRAIL IL-6, YKL-40, ICAM-1, VCAM-1, NfL, A1AT, TTR, Neurogranin, and/or hFABP even in the presence of human saliva and forms an antigen-binding agent complex. Then the presence and amount of the complex is detected using a second binding agent linked to a detectable reporter. In some embodiments, the specific binding agent and the second binding agent are the same. In the case of binding reagents that are polyclonal antibodies, the same preparation of polyclonal antibodies may be used for both antigen-binding and the detectable reporter.

In one embodiment of a kit, the binding agent is an antibody or an antigen binding part thereof comprising an immunoglobulin domain known in the art, or another specific binding agent known in the art such as a ligand, receptor, chemical ligand, aptamer, etc.

In one embodiment, a diagnostic kit capable of performing assaying methods for detection and quantification of one or more biomarkers in a saliva sample indicative of AD could comprise an ELISA-type assay. ELISA assays are well suited for detection of biomarkers such as the salivary proteins described herein. In one form of an ELISA assay, saliva containing one or more of IGFBP-2, IGFBP-3, BACE1, GSH, TRAIL, IL-6, YKL-40, ICAM-1, VCAM-1, NfL, A1AT, TTR, Neurogranin, and/or hFABP may be attached to a substrate such as a microtiter plate. Antibodies or aptamers specific to a biomarker may be applied over the surface for binding to any biomarkers which may be present in the saliva. Such antibodies or aptamers could be considered indirectly secured to the substrate provided by the microtiter plate. Such antibodies or aptamers are linked to an enzyme and then any unbound antibodies or aptamers are removed. In a final step, a substance containing the enzyme's substrate is added. If the biomarker is present in the saliva sample, there is binding and the subsequent reaction produces a detectable signal, most commonly a color change. The concentration of the biomarker can be quantified using a cutoff level or a reference value or amount.

A multiplex ELISA assay may comprise an assay capable of detecting more than one type of biomarker. Detection of one or more type of biomarker by means of the multiplex assay may be deemed indicative of AD in the subject.

In a sandwich form of ELISA capable of use in kit form, select antibodies (or aptamers) with an affinity for a type of target biomarker can be linked to the substrate of the microtiter plate. Such antibodies could be considered directly secured to the substrate provided by the microtiter plate. Saliva potentially containing target biomarkers may be applied over the substrate. Following rinsing, a fluid with labeled antibodies with affinity for the target biomarker is applied over the substrate. The label can elicit a signal when a signaling reagent is applied. The signaling reagent is capable of providing a measurable signal proportional to the concentration of the target biomarker present on the substrate. A multiplex ELISA assay may comprise an assay capable of detecting more than one type of biomarker.

The kit may include instructions describing how to use the kit. In embodiments the instructions may explain steps such as how to collect a saliva sample, how much saliva is necessary, how to load the saliva onto the assay, what to do after the saliva is loaded, the time duration for the assay, and how to interpret the results.

Kits and assays according to the invention have the capability of detecting biomarkers indicative of AD at a very early stage of the disease process. Such early stage detection is particularly useful in identifying subjects who are asymptomatic of AD and who would otherwise not be aware of the onset of the disease. Early detection of AD, of course, provides opportunities for early clinical intervention.

In another embodiment, the present specification provides for an immunoassay which may be in the form of a kit for point-of-care use to assess AD in a saliva sample from a subject. The assay may comprise: a specific binding agent that specifically recognizes an epitope of one, or a combination of, IGFBP-2, IGFBP-3, BACE1, GSH, TRAIL, IL-6, YKL-40, ICAM-1, VCAM-1, NfL, A1AT, TTR, Neurogranin, and/or hFABP or a metabolite thereof, that is not efficiently or substantially recognized by rodent/mouse antibodies or other antibodies when the enzyme or metabolite are present in the saliva to form an antigen/epitope-binding agent complex and detecting the complex using a second or further binding agent linked to or comprising a detectable reporter. The assay enables detecting of AD in the subject when the concentration or level or activity of the one or combination of more than one of, IGFBP-2, IGFBP-3, BACE1, GSH, TRAIL, IL-6, YKL-40, ICAM-1, VCAM-1, NfL, A1AT, TTR, Neurogranin, and/or hFABP or metabolite exceeds a recognized reference level. A measurement device may be implemented to accomplish the detection in certain embodiments. Iterations of a measurement device may be operable, for example, to utilize a label associated with the binding agent to provide a qualitative, semi-quantitative, or quantitative measure of the one or more biomarker in the saliva sample indicative of whether the asymptomatic subject is afflicted with AD.

The specific binding agent may comprise a rabbit antibody or an antigen binding part thereof. Alternatively, another binding agent may be implemented which recognizes the same epitope as the rabbit antibody. The binding agent may be an antibody or an antigen-binding fragment thereof, an antigen-binding construct such as an aptamer, or a ligand or binding part thereof. In embodiments including a biomarker panel or panels, the reagents or binding agents may comprise one or more binding agent which specifically binds to the biomarkers of the biomarker panels. Preferably, the one or more binding agents are primary antibodies and each primary antibody may specifically bind to a different biomarker of the biomarker panel. The reagents may further comprise one or more secondary antibodies which specifically bind to said primary antibodies. Optionally, the secondary antibodies are labeled.

In one embodiment, the assay is an enzyme-linked immunosorbent (ELISA)-type or immunochromatographic-type assay and the specific binding agent is immobilized on a support. In another embodiment, the assay may be a microfluidic-type assay known in the art.

In such embodiments, the saliva sample may be brought into contact with the binding agents by applying the saliva sample to a sample portion of an immunochromatographic or microfluidic device. The sample portion of the device may be operably connected to spaced capture portions of the device. The saliva sample is allowed to flow from the device sample portion to and through the device capture portions. The capture portion may further include the binding agent which then specifically binds to the antigen in the saliva sample as it flows past. In one embodiment, the capture portion is a test line. The antigen (i.e., the biomarker) is captured by the binding agent to form a binding agent-antigen complex in the capture portion.

In one embodiment, the amount of antigen complex is detected using binding agents such as an antibody or antigen-binding fragment, ligand or aptamer that specifically binds the antigen and directly or indirectly provides a detectable signal that can be quantified visually or photometrically including fluorometrically, for example by a measurement device capable of detecting and providing a qualitative, semi-quantitative, or quantitative measure of the one or more biomarker in the saliva sample. That information can be utilized to determine whether the subject is, or is not, afflicted with AD.

In one embodiment, the specific binding agents are conjugated to a detectable marker or microparticles comprising a detectable marker, that provide a detectable signal. Such detectable signal may be detected with a measurement device.

In one embodiment, the visual or photometrically calculated signal from the biomarker test line is used to determine and quantify the concentration of the one or combination of more than one of IGFBP-2, IGFBP-3, BACE1, GSH, TRAIL, IL-6, YKL-40, ICAM-1, VCAM-1, NfL, A1AT, TTR, Neurogranin, and/or hFABP present in the subject's saliva sample.

In yet another embodiment, the present invention provides a kit for measuring the mass concentration of one or more of IGFBP-2, IGFBP-3, BACE1, GSH, TRAIL, IL-6, YKL-40, ICAM-1, VCAM-1, NfL, A1AT, TTR, Neurogranin, and/or hFABP for AD. In such embodiments, the kit may include a chromatographic device comprising a porous membrane operably connected to a sample portion, one or more capture (test) portions, and optionally one or more of the following: a conjugate (detection marker) portion, an absorption pad portion, a suitable control portion and, optionally, a cell lysis or solubilization portion.

The capture portion may include a lagomorph antibody that recognizes an epitope of one or more of IGFBP-2, IGFBP-3, BACE1, GSH, TRAIL, IL-6, YKL-40, ICAM-1, VCAM-1, NfL, A1AT, TTR, Neurogranin, and/or hFABP and forms a complex with one or more of IGFBP-2, IGFBP-3, BACE1, GSH, TRAIL, IL-6, YKL-40, ICAM-1, VCAM-1, NfL, A1AT, TTR, Neurogranin, and/or hFABP. A labeled rabbit antibody complex or a labeled binding agent comprising the antigen binding component thereof may be provided for detection of the biomarker. Instructions for using the device and to interpret the results are preferably included with the kit. In one embodiment, the device may be suitable for reverse or lateral flow immunochromatographic formats.

In another embodiment, the present invention provides a kit comprising a chromatographic device. The chromatographic device may include a porous membrane or a microfluidic device operably connected to a sample portion, two or more capture portions, and optionally one or more of the following: a conjugate portion, an absorption pad portion, a suitable control portion and, optionally, a cell lysis or solubilisation portion.

The capture portion may include a specific lagomorph antibody or binding agent that comprises the antigen binding part thereof or specific binding agent that recognizes an epitope of an enzyme or metabolite, such as one that is not recognized by rodent antibodies when the enzyme or metabolite are present in saliva, and forms an IGFBP-2, IGFBP-3, BACE1, GSH, TRAIL, IL-6, YKL-40, ICAM-1, VCAM-1, NfL, A1AT, TTR, Neurogranin, and/or hFABP enzyme-antibody/binding agent complex. A second or secondary binding agent may be provided that binds specifically to IGFBP-2, IGFBP-3, BACE1, GSH, TRAIL, IL-6, YKL-40, ICAM-1, VCAM-1, NfL, A1AT, TTR, Neurogranin, and/or hFABP in the sample and forms IGFBP-2, IGFBP-3, BACE1, GSH, TRAIL, IL-6, YKL-40, ICAM-1, VCAM-1, NfL, A1AT, TTR, Neurogranin, and/or hFABP marker-binding agent complex wherein the binding agents are either immobilized to separate capture portions and/or contained within conjugate portions. Examples of secondary binding agents may include antibodies, antigens, and nanoparticles, aptamers, inhibitors, substrates, cofactors, coenzymes, lectins, nucleic acids, protein A, protein G, nonbiological ligands, boronates, triazine dyes, metal-ion chelates, etc. Optionally, user instructions are provided providing direction to the user for proper performance of the process for detection and evaluation of one or more of IGFBP-2, IGFBP-3, BACE1, GSH, TRAIL, IL-6, YKL-40, ICAM-1, VCAM-1, NfL, A1AT, TTR, Neurogranin, and/or hFABP as a measure of AD.

In one embodiment the kit is a reverse or lateral flow immunochromatographic format.

In one embodiment, the binding agent comprises an immunoglobulin domain.

In accordance with another iteration of the invention, a low-cost, point-of-care, screening kit may be provided for use by anyone with minimal training to screen saliva samples for the presence of biomarkers indicative of AD. A benefit of such a kit is the opportunity to detect AD at an earlier stage which, if left untreated, can result in severe AD complications. Such a kit may include a plurality of reagents and a substrate. The substrate may have an outer surface that is configured to receive one of the reagents and react with it so as to cause the substrate's outer surface to acquire a first characteristic color. Following color formation, a saliva sample may be introduced over a portion of the substrate containing the reagent. As a result of the formulation of each of the reagents, a second, dichotomous characteristic color forms if the selected biomarker or biomarkers indicative of AD are present in the saliva. The presence of the second characteristic color associated with the substrate is indicative that the subject's saliva is positive for the selected biomarker or biomarkers potentially indicative of AD.

This screening kit may also include a plurality of containers and container closures. Each container may have an outer surface that includes an orifice and is configured to receive, store and dispense a prescribed quantity of one the plurality of reagents. Each closure may be configured to cover the orifice of one the containers. As an aid to proper use, each outer surface of each container and the corresponding closure may be configured with indicia indicative of the reagent within the container and biomarker which is the subject of the screening. In such example, the characteristic first and second colors are indicative of the individual having been screened for AD.

The methods, kits, and systems described herein include determination of salivary biomarker reference levels that may be utilized to determine whether any subject, or group of subjects, is or may be afflicted with AD as well as to determine a level of severity of AD affliction. The reference levels may be based on levels of biomarkers in the saliva of individuals who are either healthy, or who are known to be afflicted with AD, or who are known to be afflicted with AD by a specific measurable score of severity of AD such as can be established through MMSE and/or CDR evaluation. Such reference levels may be from a population of individuals who are cognitively normal, a population of individuals who have been diagnosed with AD, a population of individuals who have been diagnosed with specific measurable severity of AD, or even a saliva sample taken at a prior date from the subject currently being evaluated for the existence and/or severity of AD affliction. These reference levels may then provide a basis for comparison to determine whether the amount of biomarker detected in the subject's saliva is indicative that the subject is at risk for AD, is afflicted with AD, or has a specific level of severity of AD.

The biomarker reference levels may be expressed in any suitable units. For example, the user may determine biomarker concentrations in either mass concentration units or equivalent enzyme level units. Reference levels may be also be expressed as ratios, or levels as appropriate.

The level of the biomarker or biomarkers may encompass the level of protein concentration or the level of biomarkers. In either iteration, the level is quantified such that a value, an average value, mean and standard deviation, or a range of values may be determined. In one embodiment, the level of protein concentration of the AD biomarker or biomarkers is/are quantified.

The skilled person will appreciate that reference levels and suitable controls are determined using standard approaches as a routine development task. Thus, for example, a comparison of one, or a combination of, IGFBP-2, IGFBP-3, BACE1, GSH, TRAIL, IL-6, YKL-40, ICAM-1, VCAM-1, NfL, A1AT, TTR, Neurogranin, and/or hFABP mass concentration levels, or enzyme levels, or activities, may be between subjects or groups of subjects to determine reference levels. A test result may be compared with reference levels for healthy subjects or with controls who have AD and thus higher or lower comparative levels can be expected depending upon the reference level selected.

In one embodiment, measurement of levels of the AD biomarkers such as one, or a combination of, IGFBP-2, IGFBP-3, BACE1, GSH, TRAIL IL-6, YKL-40, ICAM-1, VCAM-1, NfL, A1TA, TTR, Neurogranin, and/or hFABP in the saliva sample of a subject being evaluated for AD affliction may be correlated with a reference level of the one, or more than one of IGFBP-2, IGFBP-3, BACE1, GSH, TRAIL, IL-6, YKL-40, ICAM-1, VCAM-1, NfL, A1AT, TTR, Neurogranin, and/or hFABP level or activity for a subject who is not afflicted with AD. The reference level or value can be obtained by measuring an amount of the biomarker in age-matched control subjects who are not afflicted with AD (i.e., are free of AD).

In yet another embodiment, measurement of levels of the AD biomarkers such as IGFBP-2, IGFBP-3, BACE1, GSH, TRAIL, IL-6, YKL-40, ICAM-1, VCAM-1, NfL, A1AT, TTR, Neurogranin, and/or hFABP (separately or in combination) in the saliva sample of a subject being evaluated for the severity of AD affliction may be correlated with the IGFBP-2, IGFBP-3, BACE1, GSH, TRAIL, IL-6, YKL-40, ICAM-1, VCAM-1, NfL, A1AT, TTR, Neurogranin, and/or hFABP level or activity for subjects with different levels of severity of AD. The reference level or value can be obtained by measuring an amount of the biomarker in age-matched control subjects who have been determined to have a particular stage of AD as determined by a measurable standard such as MMSE or CDR evaluation.

In certain embodiments, very high mass concentrations or levels of IGFBP-2, IGFBP-3, BACE1, GSH, TRAIL, IL-6, YKL-40, ICAM-1VCAM-1, NfL, A1AT, TTR, Neurogranin, and/or hFABP or a metabolite thereof are diagnostic of AD.

It has been found that certain reference levels are highly predictive of whether a subject is afflicted with AD. A determination of whether the subject's salivary biomarkers exceed, or do not exceed, the threshold provided by the reference levels may be utilized for prediction, detection, monitoring, treatment, and for formation of a prognosis and diagnosis of AD in the subject. In embodiments, a subject who has some outward manifestations of AD (i.e., is deemed symptomatic of AD) may be considered positive for AD if the subject's concentration of detected salivary biomarkers meet or exceed one or more of the following criteria on a mass concentration basis: IGFBP-2 above about 2500 pg/ml, IGFBP-3 above about 1680 ng/ml, BACE1 above about 700 pg/ml, GSH less than about 1.7 μmol/l, TRAIL above about 3.0 ng/ml, IL-6 above about 20 pg/ml, YKL-40 above about 30 ng/ml, ICAM-1 above about 200 ng/ml, VCAM-1 above about 450 ng/ml, NfL above about 0.3 pg/ml, A1AT less than about 650 ng/ml, TTR less than about 10 μg/ml, a Neurogranin level above about 5 pg/ml, and hFABP above about 0.80 ng/ml.

A further advantage of the invention is that detection of certain reference levels of salivary biomarkers are highly predictive of AD in a subject who is asymptomatic of AD. Such an asymptomatic subject may be a MCIAD subject or have a lesser degree of affliction with AD. In embodiments, an asymptomatic subject may be considered positive for AD if the subject's concentration of detected salivary biomarkers meet or exceed one or more of the following criteria: IGFBP-2 within a range of about 2600 pg/ml to about 3800 pg/ml, IGFBP-3 within a range of about 2100 ng/ml to about 2600 ng/ml, BACE1 within a range of about 800 pg/ml to about 1000 pg/ml, GSH in the range of about 1 μmol/l to about 1.6 μmol/l, TRAIL in the range of about 3.2 ng/ml to about 5.5 ng/ml, IL-6 in the range of about 25 pg/ml to about 35 pg/ml, YKL-40 in the range of about 34 ng/ml to about 50 ng/ml, ICAM-1 in the range of about 200 ng/ml to about 320 ng/ml, VCAM-1 in the range of about 720 ng/ml to about 1000 ng/ml, NfL in the range of about 0.5 pg/ml to about 2.4 pg/ml, A1AT in the range of about 200 ng/ml to about 500 ng/ml, Neurogranin in the range of about 4.5 pg/ml to about 7 pg/ml, TTR in range of about 3.0 to about 10.1 μg/ml, and hFABP in the range of about 1.00 ng/ml to about 1.56 ng/ml.

Generally a correlation of not less than about 70% between AD biomarker levels in the subject under evaluation and the reference level will be effective to provide a confidence level that the subject either is, or is not, afflicted with AD or has a certain level of severity of AD (e.g. asymptomatic, mild, moderate, severe). Higher correlation levels of 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% are contemplated and provide a higher confidence level that the measured salivary biomarker in the subject is determinative of the state of the disease in the subject.

The present disclosure further contemplates a method of treating AD. In iterations, the method may include establishing a baseline value of the mass concentration or level of biomarker or biomarkers in the subject's saliva. The biomarkers may be one, or a combination of more than one, of IGFBP-2, IGFBP-3, BACE1, GSH, TRAIL, IL-6, YKL-40, ICAM-1, VCAM-1, NfL, A1AT, TTR, Neurogranin, and/or hFABP. The biomarker, or biomarkers, may be detected in the subject's saliva sample using assaying techniques such as LFA or ELISA. The subject's saliva sample may, for example, be contacted with a lagomorph antibody or rabbit antibody that specifically recognizes an epitope of IGFBP-2, IGFBP-3, BACE1, GSH, TRAIL, IL-6, YKL-40, ICAM-1, VCAM-1, NfL, A1AT, TTR, Neurogranin, and/or hFABP. If the biomarker is present, an antigen-antibody complex is formed and the complex may be detected using a second binding agent linked to a detectable reporter. With the baseline established, various treatment programs may be implemented for the subject. The subject's saliva may be re-assayed at appropriate decision points in the treatment program. The efficacy of the treatment program may be evaluated based on changes, or lack of changes, in the mass concentration or level the biomarker relative to the baseline.

The biomarkers IGFBP-2, IGFBP-3, BACE1, GSH, TRAIL, IL-6, YKL-40, ICAM-1, VCAM-1, NfL, A1AT, TTR, Neurogranin, and/or hFABP are stable and remain stable for a time duration that can exceed 20-30 days or a greater number of days. This stability enables retesting of the saliva sample, for example, to enable more accurate treatment regimens.

In certain embodiments, the efficacy of the treatment for AD may include measuring mass concentrations or levels of a complex including a set of the biomarkers IGFBP-2, IGFBP-3, BACE1, GSH, TRAIL, IL-6, YKL-40, ICAM-1, VCAM-1, NfL, A1AT, TTR, Neurogranin, and/or hFABP, for example in a multiplex assay. In embodiments, the efficacy of treatment is measured by monitoring levels of the set of biomarkers in the subject's saliva compared to a reference, and/or compared to other previous tests of the subject, or to an earlier stage of treatment/AD in the subject.

Biomarker panels of the types described herein may be used to select drugs and agents for the ability to prevent or treat AD, or one or more symptoms thereof. Examples of drugs and agents may be cholinesterase inhibitors (Aricept®, Exelon®, Razadyne®), memantine (Namenda®), and others.

The drugs or agents might be tested in human subjects in clinical trials. Any drug and agent which maintains or, alternatively, changes the levels of the biomarker or biomarkers described herein towards levels found in healthy individuals is of potential use in treating AD. Treatment could result in lessening of AD symptoms and/or slowing the progression of AD.

During clinical trials, for example, the amount or concentration of one or more biomarker of a biomarker panel as described herein can be estimated in the presence or absence of a drug being tested. The efficacy of the agent or drug can be followed by comparing changes in the levels of the biomarker or biomarkers corresponding to known levels of the biomarkers in a normal, non-diseased, state.

Agents exhibiting efficacy are those which alter the presence, amount, or concentration of the biomarkers in the biomarker panel to more closely resemble that of the state in which the subject is not afflicted with AD. Because the present biomarker panels translate events and changes in pathways that occur in the brain into a peripheral signal, they allow replacing tissue testing with bodily fluid testing, preferably a saliva sample.

An agent or drug might be selected if it prevents or slows the change over time in presence, concentration, or amount of the biomarkers of the biomarker panels relative to controls. Preferably, the agent or drug is selected if it converts the amount or concentration of a biomarker of the biomarker panels or biomarker towards that of a normal subject who is not afflicted with AD.

The agent might be selected if it slows, or stops, or moves toward a normal value of the change of concentration or amount over time. For example, agents which exhibit inhibitory activity, might be used in accordance with the invention to prevent mild cognitive impairment or AD symptoms. Such molecules may include, but are not limited to, peptides, phosphopeptides, small organic or inorganic molecules, or antibodies, herbal agents, etc.

In another embodiment, the present disclosure provides a method of screening for binding agents that are substantially not inhibited from antigen binding by saliva. Such a method may comprise contacting an antigen and a potential antigen binding agent in the presence of different concentrations of saliva to determine whether the binding agent can bind the antigen in the presence of saliva. In one embodiment, the binding agent is an antibody, a monoclonal antibody or an antigen binding part thereof, a peptide ligand, and/or a nucleic acid binding ligand.

There has been a lack of understanding of salivary forms of the biomarkers and biomarker combinations as described and claimed herein and their correlation with AD and, especially asymptomatic AD. This is because no studies have been conducted on salivary biomarkers as claimed in the early detection, diagnosis, prognosis, and monitoring of AD. The biomarkers may be better understood by means of the following description.

The biomarkers IGFBP-2 and IGFBP-3 are early precursors within the insulin-like growth factor (IGF) signaling system. The IGF signaling system is known to be associated with important functions in cerebral metabolic function, neuroregeneration, neuronal survival, and proliferation. Fernandez, A. M., et al., The Many Faces of Insulin-Like Peptide Signalling in the Brain, Nat. Rev. Neurosci. 13:225-239 (2012). Circulating IGF-I and IGF-II are characteristically bound to one of six well-known IIGF-binding proteins. As IGFBP-2 is the most important IGF-binding protein, it is also the most predominant IGF-binding protein in the brain. Hertze, J., et al., Changes in Cerebrospinal Fluid and Blood Plasma Levels of IGF-II and its Binding Proteins in Alzheimer's Disease: An Observational Study, BMC Neurol. 14:64 (2014). Increased levels of IGF-binding proteins, mainly IGFBP-2, are thought to decrease IGF-I and IGF-II bioavailability in the brain and impair IGF signaling, thus inhibiting the neuroprotective effects of IGF-I and IGF-II. Mackay K. B., et al., Neuroprotective Effects of Insulin-Like Growth Factor-Binding Protein Ligand Inhibitors In Vitro and In Vivo, J. Cereb. Blood Flow Metab. 23:1160-1167 (2003). Peripheral levels of both IGF-1 and IGFBP-2 have been linked with an increased risk of clinical AD. Additionally, there is growing evidence that of the six high-affinity IGF-binding proteins, IGFBP-2 might have a specific role in AD pathophysiology. Westwood, A. J., et al., Insulin-Like Growth Factor-1 and Risk of Alzheimer Dementia and Brain Atrophy, Neurology 82(18), 1613-1619 (2014); Doecke, J. D., et al., Blood-Based Protein Biomarkers for Diagnosis of Alzheimer Disease, Archives of Neurology 69:1318-1325 (2012). IGFBP-2 might drive neurodegeneration by exacerbating IGF-1 signaling defects among individuals with AD neuropathology. Toledo, J. B., et al., Relationship Between Plasma Analytes and SPARE-AD Defined Brain Atrophy Patterns in ADNI, PloS. One 8, e55531 (2013).

IGFBP-3 is considered to be the major IGF-1 carrier. Koistinen, H., et al., Effect of Marathon Run on Serum IGF-I and IGF-Binding Protein 1 and 3 Levels, J. Appl. Physiol. 80 (3): 760-4(1996). IGF-I and IGFBP-3 are concerned in oxidative stress and longevity. Holzenberger, M., et al., IGF-I Receptor Regulates Lifespan and Resistance to Oxidative Stress in Mice, Nature 421:182-187 (2003). IGF-I bioavailability is regulated via the IGFBP-3 concentration. Froesch, E. R., et al., Insulin-Like Growth Factor I: Physiology, Metabolic Effects and Clinical Uses, Diabetes Metab. Rev. 12:195-215 (1996). Prior to the present research work, no study has been conducted on salivary IGFBP-2 and IGFBP-3 levels in early detection, diagnosis, prognosis and monitoring of AD.

Amyloid betapeptide (Aβ) is a proteolytic product of the amyloid precursor protein (APP) which is consecutively cleaved in an amyloidogenic pathway by beta- and gamma-secretases. Selkoe, D. J., Alzheimer's Disease, Cold Spring Harb. Perspect. Biol. 3:a004457 (2011). Beta-secretase cleaves APP close to the membrane, releasing a soluble APP beta-fragment (sAPPb). The remaining C-terminal fragment (APP-CTFb) can then be cleaved by gammasecretase at different sites yielding Aβ-peptides of varying length, such as Aβ-40 and Aβ-42. Of these, Aβ-40 is most common, while Aβ-42 is more toxic and prone to aggregate. If APP is cleaved in a non-amyloidogenic pathway by alpha-secretase instead of beta-secretase, a non-toxic P3 fragment will be formed after the gamma-secretase cleavage. BACE1, beta-site APP-cleaving enzyme 1, has been identified in vivo as a 501-amino acid residue single transmembrane aspartyl protease. Roberds, S. L., et al., BACE Knockout Mice are Healthy Despite Lacking the Primary Beta-Secretase Activity in Brain: Implications for Alzheimer's Disease Therapeutics, Hum. Mol. Genet. 10: 1317-1324 (2001). BACE1 cleaves APP at two beta sites and produces the C-terminal fragments (CTF) C99 and C89. Sun, X., et al., BACE2, as a Novel APP Thetasecretase, is Not Responsible for the Pathogenesis of Alzheimer's Disease in Down Syndrome, FASEB. J. 20: 1369-1376 (2006). C99 is subsequently cleaved by presenilin (PS) dependent gamma-secretase complex to release Aβ fragments. BACE1, therefore, represents a potentially key target enzyme in the diagnosis, monitoring, and possible treatment of AD. Importantly, no studies to date have been conducted to demonstrate that salivary BACE1 is efficacious in early detection, diagnosis, prognosis, and monitoring of AD.

In AD, oxygen and nitrogen free radicals induce protein, lipid, and DNA oxidation which leads to cytotoxic effects on the subject. Konukoglu, D., et al., Comparing Oxidative Stress Markers and S100B, Aβ-40 Proteins as Independent Neurological Markers in Distinguishing the Relation of Alzheimer's Disease and Diabetes Mellitus, J. Neurol. Neurosci. 7(5):146 (1-10) (2016). Impaired permeability of the blood brain barrier and endothelial damage in small vessels in AD patients have been observed implying that the presence or increase of oxidative radicals or free radical in systemic circulation might affect the brain in subjects with AD. In patients with dementia, the antioxidant properties of saliva are reduced, the level of oxidative products of DNA, protein, and lipid damage is increased, which is accompanied by reduction of saliva secretion. Choromanska, M., et al., Antioxidant Defence, Oxidative Stress and Oxidative Damage in Saliva, Plasma and Erythrocytes of Dementia Patients. Can Salivary AGE Be A Marker of Dementia? Int. J. Mol. Sci. 18,2205 (2017).

Glutathion (GSH) may be useful as an oxidative stress marker. Tarkowski, E., et al., Cerebral Pattern of Pro-and Anti-Inflammatory Cytokines in Dementias, Brain Res. Bull. 15; 61(3):255-60 (2003). In transgenic murine models, the reduced form of the tripeptide thiol glutathione (GSH) is one of the most abundant intracellular antioxidants and free radical scavengers. Reduced GSH is a reliable marker of oxidative stress which has been reported to precede amyloid oligomerization and plaque formation, both pathologic hallmarks of AD. Resende, R., et al., Brain Oxidative Stress in a Triple-Transgenic Mouse Model of Alzheimer Disease, Free Radic. Biol. Med. 44:2051-57 (2008). A self-propagating cycle of free radical formation, oxidative stress, and amyloid plaque formation has also been shown in vitro. Hensley, K. et al., A Model for Beta-Amyloid Aggregation and Neurotoxicity Based on Free Radical Generation by the Peptide: Relevance to Alzheimer's Disease, Proc. Natl. Acad. Sci. USA 91:3270-74 (1994). Importantly, however, no study has yet been conducted on salivary GSH levels in the early detection, diagnosis, prognosis, and monitoring of AD. Inflammatory mechanisms have been strongly linked to the pathogenesis of AD. Cytokines involved in the inflammatory process located close to amyloid plaques might be cytotoxic when produced chronically and might stimulate the production of Aβ peptides. Increased oxidative stress markers were found in brains of amnestic mild cognitive impairment subjects, most of whom with pre-AD. Keller, J. N., et al., Evidence of Increased Oxidative Damage in Subjects with Mild Cognitive Impairment, Neurology 64:1152-1156 (2005). Chronic inflammation was proposed as a dysregulated mechanism in AD patients. Krstic, D., et al., Deciphering the Mechanism Underlying Late-Onset Alzheimer Disease, Nat. Rev. Neurol. 9:25-34 (2012). In hippocampal neurons, Aβ and IL-6 were both capable of inducing synaptic dysfunction. Lee, K. S., et al., Peripheral Cytokines and Chemokines in Alzheimer's Disease, Dement. Geriatr. Cogn. Disord. 28:281-287 (2009). Different studies attempted to prove the validity of IL-6 levels in the serum or CSF as a biomarker for AD. Nevertheless the results were inconclusive. Anoop, A., et al., CSF Biomarkers for Alzheimer's Disease Diagnosis, Int. J. Alzheimer's Dis. 2010:606802 (2010). Importantly, no study to date has been conducted on the biomarker combinations described herein which include assessment of IL-6 levels in the early detection, diagnosis, prognosis, and monitoring of AD.

TRAIL, the tumor necrosis factor-related apoptosis-inducing ligand, has a dual role in inflammation. The TRAIL receptor could mediate oligomeric Aβ-induced apoptosis and pro-survival signaling pathways. Fossati, S., et al., TRAIL Death Receptors DR4 and DR5 Mediate Cerebral Microvascular Endothelial Cell Apoptosis Induced by Oligomeric Alzheimer's Aft Cell Death Dis. 3:e321 (2012). TRAIL could activate caspases to cleave Beclin-1 and Atg5, thus leading to cytotoxicity. Salminen, A., et al., Impaired Autophagy and APP Processing in Alzheimer's Disease: the Potential Role of Beclin 1 Interactome, Prog. Neurobiol. 106-107:33-54 (2013). Neutralization of TRAIL can protect Aβ-induced toxicity in vitro. Cantarella, G., et al., Neutralization of TRAIL Death Pathway Protects Human Neuronal Cell Line From Beta-Amyloid Toxicity, Cell Death Differ. 10:134-141 (2003). No study has yet been conducted on salivary TRAIL levels in early detection, diagnosis, prognosis and monitoring of AD. Pathogenesis of AD is not limited to the neuronal compartment, but also involves various immunological mechanisms. Insoluble Aβ aggregates in the brain can induce the activation of microglia, resulting in the synthesis of proinflammatory mediators, which can further stimulate astrocytic expression of YKL-40. Rosén, C., et al., Increased Levels of Chitotriosidase and YKL-40 in Cerebrospinal Fluid from Patients with Alzheimer's Disease, Dement. Geriatr. Cogn. Dis. Extra 4:297-300 (2014). There has yet to be any study of salivary YKL-40 levels in early detection, diagnosis, prognosis and monitoring of AD.

ICAM-1 and VCAM-1 are adhesion molecules that are upregulated in endothelial cells under inflammatory conditions. Lee, S. J., et al., Adhesion Molecule Expression and Regulation on Cells of the Central Nervous System, J. Neuroimmunol. 98:77-88 (1999). In postmortem tissue from patients with AD, increased expression of ICAM-1 has been detected in plaques and astrocytes around plaques. Akiyama, H., et al., Expression of Intercellular Adhesion Molecule (ICAM)-1 by a Subset of Astrocytes in Alzheimer Disease and Some Other Degenerative Neurological Disorders, Acta. Neuropathol. 85:628-634 (1993). High levels of ICAM-1 in temporal and cingulate cortices have been shown to correlate with larger Aβ0 plaque and neurofibrillary tangle loads in Lewy body disorder. Walker, D. G., et al., Changes in CD200 and Intercellular Adhesion Molecule-1 (ICAM-1) Levels in Brains of Lewy Body Disorder Cases are Associated with Amounts of Alzheimer's Pathology Not Alpha-Synuclein Pathology, Neurobiol. Aging 54:175-186 (2017). But no study was conducted on salivary ICAM-1 and VCAM-1 levels in early detection, diagnosis, prognosis and monitoring of AD.

Neurofilament light chain (NfL) represents a potential serum-based biomarker of neural damage currently widely evaluated in a number of neurological conditions, Khalil, M., et al., Neurofilaments as Biomarkers in Neurological Disorders, Nat. Rev. Neurol. 14:577-89 (2018). NfL is a cytoskeleton protein expressed in large caliber myelinated axons, and it is released in the extra-cellular fluid as consequence of axonal damage. Thus, while other fluid markers such as beta-amyloid and tau are aimed to help in the diagnosis of a specific clinical conditions (i.e., AD), NfL is instead a trans-diagnostic marker of neurodegeneration. NfL in serum and CSF has been associated with a number of different neurological conditions and sensitive commercially-available assays exist to detect NfL in serum and CSF. Rissin, D. M., et al., Single-Molecule Enzyme-Linked Immunosorbent Assay Detects Serum Proteins at Subfemtomolar Concentrations, Nat. Biotechnol. 28:595-9 (2010). NfL is thought to be an attractive putative serum-based marker of neurodegeneration in AD subjects. Preische, O., et al., Serum Neurofilament Dynamics Predicts Neurodegeneration and Clinical Progression in Presymptomatic Alzheimer's Disease, Nat. Med. 25:277-83 (2019). But no study was conducted on salivary NfL levels in early detection, diagnosis, prognosis and monitoring of AD.

Astrocytes are the major type of cell responsible for production of A1AT because A1AT protein is localized in these types of cells and astrocytes are involved in Alzheimer's disease lesions. Gollin, P. A., et al., A1AT and Alpha 1-Antichymotrypsin are in the Lesions of Alzheimer's Disease, Neuroreport. February, 3(2):201-3 (1992). One treatment and management approach for AD is to modify neuroinflammation towards an activation state that is differentiated by reduced production of pro-inflammatory mediators and increased Aβ clearance. Heneka, M. T., et al., NLRP3 is Activated in Alzheimer's Disease and Contributes to Pathology in APP/PS1 Mice, Nature 493: 674-678 (2013). Recently, a new anti-inflammatory role for A1AT has been described for activated monocytes as well as for lung endothelial cells. Lockett, A. D., et. al., Alpha(1)-Antitrypsin Modulates Lung Endothelial Cell Inflammatory Responses to TNF-Alpha, Am. J. Respir. Cell Mol. Biol. 49: 143-150 (2013). But no study has yet been conducted on salivary A1AT levels in early detection, diagnosis, prognosis, and monitoring of AD.

TTR is believed to inhibit amlyloid formation in normal cerebrospinal fluid (CSF). The TTR inhibits function of the major A-Beta binding protein in CSF, leading to a decrease in the aggregation state of the peptide. Schwarzman, A. L., et al. TTR Sequesters Amyloid Beta Protein and Prevents Amyloid Formation, Proceedings of the National Academy of Sciences of the United States of America 91: 8368-8372 (1994). The TTR/A-Beta interaction indicates that TTR is capable of interfering with A-Beta fibrillization by both inhibiting and disrupting fibril formation. Costa R, et al., TTR Binding to A-Beta Peptide-Impact on A-Beta Fibrillogenesis and Toxicity, FEBS Letters 582: 936-942 (2008). TTR, when mutated, is linked to another amyloidotic disorder, Familial Amyloid Polyneuropathy (FAP), characterized by the extracellular deposition of TTR in several organs with a special emphasis in the peripheral nerve. Ferreira, N., et al., Natural Polyphenols Inhibit Different Steps of the Process of TTR Amyloid Fibril Formation, FEBS Letters 585: 2424-2430. But no study has yet been conducted to determine whether salivary TTR levels may be efficacious in the early detection, diagnosis, prognosis, and monitoring of AD.

Neurogranin is also known as RC3, canarigranin, B-50-immunoreactive C-kinase substrate and p17. It is a 78 amino acid-long post-synaptic protein. Represa, A., et al., Neurogranin: Immunocytochemical Localization of a Brainspecific Protein Kinase C Substrate, J. Neurosci. 10, 3782-3792 (1990). Neurogranin expression is altered in AD, where it may be implicated in cognitive impairment. Kovalevich, J., et al., Cocaine Decreases Expression of Neurogranin Via Alterations in Thyroid Receptor/Retinoid X Receptor Signaling, J. Neurochem. 121, 302-313 (2012). Neurogranin was significantly associated with the degree of amyloid and tau pathology. Processing of Neurogranin into peptides is increased in AD brain tissue, which may reflect the ongoing synaptic degeneration, and which is also mirrored as increased levels of Neurogranin peptides in CSF. Tarawneh, R., et al., Diagnostic and Prognostic Utility of the Synaptic Marker Neurogranin in Alzheimer Disease, JAMA Neurol. 73:561-571 (2016). But no study has yet been conducted on salivary Neurogranin levels in early detection, diagnosis, prognosis and monitoring of AD.

Human heart fatty acid binding protein, hFABP, functions in the intracellular lipid metabolism by carrying fatty acids, and elevated CSF levels of this protein have been reported in studies on dementia and Creutzfeldt-Jakob disease. Steinacker, P., et al., Heart Fatty Acid Binding Protein as a Potential Diagnostic Marker for Neurodegenerative Diseases, Neurosci. Letters 370:36-39 (2004). It has been found that a strong relationship exists between hFABP and a number of apolipoproteins including ApoE, as well as an interaction between hFABP and ApoC III on volume loss. Together, these findings support the hypothesis that intra-cranial lipid biology might influence Alzheimer's neurodegeneration. Di Paolo, G., et al., Linking Lipids to Alzheimer's Disease: Cholesterol and Beyond, Nat. Rev. Neurosci. 12:284-296 (2011). But no study was conducted on salivary hFABP levels in early detection, diagnosis, prognosis and monitoring of AD.

Methods, systems, and kits according to the invention may have some or all of the following advantages:

Broad applicability/high diagnostic value: Methods, systems, and kits according to the invention have a high sensitivity for, and are capable of identifying, a broad range of biomarkers indicative of AD in saliva samples. Biomarker examples are IGFBP-2, IGFBP-3, BACE1, GSH, TRAIL, IL-6, YKL-40, ICAM-1, VCAM-1, NfL, A1AT, TTR, Neurogranin, and/or hFABP, separately and in combination.

Availability of components/low cost: In addition, antibodies capable of binding to, and detecting, the target salivary biomarker antigens IGFBP-2, IGFBP-3, BACE1, GSH, TRAIL, IL-6, YKL-40, ICAM-1, VCAM-1, NfL, A1AT, TTR, Neurogranin, and/or hFABP are readily available, providing opportunities for a lower cost assay. Specifically, high-purity monoclonal or polyclonal antibodies to these biomarkers can be produced easily from mouse or rabbit sources. These detection antibodies are capable of being coated with colloidal gold and other labels for ease of identification in many different assays according to the invention.

Rapid results/ease of use: Methods, systems, and kits according to the invention are capable of detecting the target salivary biomarkers and providing results potentially within about 2 to about 30 minutes after starting the assay. In embodiments based on LFA assays, the user merely places saliva on the sample pad and waits for the result.

Methods, systems, and kits according to the invention can be portable and utilized and implemented wherever needed. Laboratory equipment and technical training are unnecessary. Methods, systems, and kits according to the invention, are suitable for clinical and home use, can quickly screen patients, and are suitable for on-site general screening and epidemiological investigation.

High accuracy: Methods, systems, and kits according to the invention yield reproducible and accurate results using many different types of assays such as LFA and ELISA assays.

Good stability: The saliva used with methods, systems, and kits according to the invention is stable. The saliva can be stored at −10° C. to 50° C. for months providing an opportunity to conduct multiple assays on the same sample over time which can be useful to verify the severity and progress of AD in a subject.

Noninvasiveness: Saliva is easily harvested from a subject and saliva collection is far less invasive to a subject than is collection of blood or another fluid or substance.

It can be appreciated that methods, systems, and kits according to the invention provide opportunities for improvements in healthcare for individual subjects, and groups of subjects, with respect to detection, diagnosis, monitoring, and treatment of AD and potentially other types of dementia-related illnesses.

### EXAMPLES

The following studies and the examples and data are provided to illustrate the invention, but are not intended to limit the scope of the invention in any way.

### Example 1

### Efficacy of Salivary Biomarkers in Prediction, Detection, Treatment and/or Monitoring of AD

Example 1 was conducted to evaluate and identify certain biomarkers and biomarker combinations in a subject indicative that the subject is afflicted with AD. A diagnosis may be made based on this information and treatment may be provided accordingly. Specifically, Example 1 sought to determine the concentrations of IGFBP-2, IGFBP-3, BACE1, GSH, TRAIL, IL-6, YKL-40, ICAM-1, VCAM-1, NfL, A1AT, TTR, Neurogranin, and/or hFABP in saliva existent in groups of healthy (i.e., control) subjects and groups of subjects who had been confirmed as testing positive for AD. Differences in the concentration of the aforementioned biomarkers between healthy and AD subjects are indicative of affliction with the disease.

As indicated in Table 1, the study was conducted with forty eight (48) subjects who had been diagnosed as being afflicted with AD. Also as indicated in Table 1, the study included a control group consisting of fifty (50) healthy non-demented control subjects who had been previously determined to lack any neurological disease or cognitive impairment. All subjects in the AD group included in these series were diagnosed with dementia according to the Diagnostic and Statistical Manual of Mental Disorders (DSM)-IV criteria (American Psychiatric Association: DSM-IV. Diagnostic and Statistical Manual of Mental Disorders. 1994, Washington D.C.: American Psychiatric Association), and NINCDS-ADRDA criteria. McKhann, G., et al. Clinical diagnosis of Alzheimer's Disease: report of the NINCDS-ADRDA Work Group under the auspices of Department of Health and Human Services Task Force on Alzheimer's Disease, Neurology 34: 939-944 (1984). Diagnosis required evidence of cognitive decline, neuropsychological test battery, clinical mental examination, as well as evidence of impairment in social or occupational function. The previously described mini-mental state examination (MMSE) was used to assess cognitive function. The mean value of MMSE score for the AD patients was 17. All subjects had an extensive biochemical evaluation including levels of vitamin B12 and folate and thyroid hormones. Each of vitamin B12 and folic acid are measured because low levels of each can result in false positives for AD.

The subjects were further evaluated with brain MRI and CT scans. Classification of mild, moderate and severe degrees of AD was performed, and the diagnosis of vascular dementia was excluded in all cases, using DSM-III-R criteria.

The control group was selected from family members of the AD subjects who were screened to ensure they had no history of AD. Each member of the control group had a clinical interview with a senior neurologist and was determined to have a completely normal cognitive and functional level. Demographic characteristics of the AD and control groups are shown in Table 1.

As is shown in Table 1, the mean age of the AD and control subjects was 73.4 years with a standard deviation (SD) of 12.3/11.6 respectively and the AD and control groups were essentially evenly divided by gender. (In the tables which follow, standard deviation is referred to as (SD)). The mean MMSE value of the AD group was 14 with a standard deviation of 5. It was determined that the mean number of years since onset of AD for the AD group had been 2.6 years with a standard deviation of 1.4. Therefore, the AD group generally represented subjects within the “moderate” dementia category according to the MMSE scale.

TABLE 1
Demographics and Baseline Characteristics of the Subjects

| | Mean Age | | | |
|---|---|---|---|---|
| | (Years)(SD), | | | Years of |
| | Age Range | Gender | MMSE Score | Onset of AD |
| | (Years) | (Men:Women) | Mean (SD) | Mean (SD) |
| AD | 73.4 | 23:25 | 14(5) | 2.6 (1.4) |
| Subjects | (12.3) | | | |
| | (61-89) | | | |
| Control | 73.4 | 25:25 | − | − |
| Subjects | (11.6) | | | |
| | (58-91) | | | |

Saliva samples were collected from each subject in the AD and control groups by means of an unstimulated drooling method in the following manner. Care was taken to ensure that all samples were collected within the same time of day window and in the same manner. Saliva samples were taken from the study participants by an oral physician on the day of testing from 9:00 AM to 10:00 AM. Individuals were asked to abstain from eating for at least two hours prior to sample collection.

10-15 minutes prior to collection of unstimulated saliva samples, individuals were asked to rinse orally with water. At the time of sample collection, study members were asked to relax for 5-15 minutes. They were then seated in a bent forward position in an ordinary chair and asked to put their tongues on the lingual surfaces of the upper incisors and to allow the saliva to drip into sterile plastic (glass) tubes treated with 50 g of 2% sodium azide solution to prevent microbial decomposition of saliva. The tubes were held to the lower lip for 10 minutes resulting in a collection of 1-5 ml of saliva per individual. Saliva samples were then centrifuged using a Sorvall RT6000D centrifuge (Sorvall, Minn.) at 1800 rpm for 5 minutes to remove debris and were then immediately frozen at −80° C., awaiting further analysis.

Various commercially available assays were implemented to determine the presence of the salivary biomarkers IGFBP-2, IGFBP-3, BACE1, GSH, TRAIL, IL-6, YKL-40, ICAM-1, VCAM-1, NfL, A1AT, TTR, Neurogranin, and hFABP. The following ELISA kits were utilized:
1. IGFBP-2 (Thermo Fisher Scientific, USA)
2. IGFBP-3 (Active IGFBP-3 Aviscera Bioscience Inc., Santa Clara, Canada)
3. BACE1 Human Beta site APP cleaving enzyme 1 (Sun Long Biotech, China)
4. GSH (Glutathione Colorimetric Detection Kit, Thermo Fisher Scientific, USA)
5. TRAIL (Thermo Fisher Scientific, USA)
6. IL-6 (Biolegend, San Diego, Calif., USA)
7. YKL-40 ELISA (R&D Systems, Minneapolis, Minn.)
8-9. ICAM-1 and VCAM-1 (R&D Systems, Minneapolis, Minn., USA)
10. NfL (Quanterix SIMOA kits, USA)
11. A1AT (Thermo Fisher Scientific, USA)
12. TTR (Aviva Systems Biology Corporation, San Diego, USA)
13. Neurogranin (American Research Products—Cloud Clone Corp.)
14. hFABP (Human heart fatty acid binding protein Duo set (Cat #DY1678), R and D systems USA)

As described in the analysis below, “area under the curve” (AUC) using a receiver operating characteristic analysis was also implemented to determine the screening ability of the salivary biomarkers to predict AD and determine the severity of the AD. Stated differently, the area under the receiver operating characteristic curve (AUC) was calculated for determining the prognostic accuracy of the salivary biomarkers. Data were analyzed by using Statistical Package for the Social Sciences and Prism (SPSS version 20 and Prism; IBM Corporation, Armonk, N.Y.). Values with P<0.05 were considered significant.

TABLE 2
Salivary Biomarker Comparison in AD and Control Groups

| | Biomarkers | | Control Subjects | | AD Patients | |
|---|---|---|---|---|---|---|
| | (Concentration) | | Mean (SD) | | Mean (SD) | |
| 1 | IGFBP2 | (pg/ml) | 2200 | (252) | 3164 | (423) |
| 2 | IGFBP-3 | (ng/ml) | 1224 | (435) | 2346 | (206) |
| 3 | BACE1 | (pg/ml) | 567 | (102) | 932 | (109) |
| 4 | GSH | (μmol/L) | 2.1 | (0.4) | 1.2 | (0.3) |
| 5 | TRAIL | (ng/ml) | 2.4 | (1.6) | 4.5 | (1.3) |
| 6 | IL-6 | (pg/ml) | 15.7 | (2.4) | 28.5 | (3.6) |
| 7 | YKL-40 | (ng/ml) | 25.8 | (9.3) | 37.3 | (6.8) |
| 8 | ICAM-1 | (ng/ml) | 152 | (54) | 218 | (48) |
| 9 | VCAM-1 | (ng/ml) | 421 | (103) | 854 | (132) |
| 10 | NfL | (pg/ml) | 0.3 | (0.2) | 1.2 | (0.4) |
| 11 | A1AT | (ng/ml) | 1567 | (453) | 367 | (103) |
| 12 | TTR | (μg/ml) | 19.4 | (3.6) | 6.2 | (3.1) |
| 13 | Neurogranin | (pg/ml) | 4.2 | (0.6) | 6.7 | (1.2) |
| 14 | hFABP | (ng/ml) | 0.68 | (0.16) | 1.32 | (0.25) |

Table 2 provides mean concentrations of the biomarkers and the standard deviation (SD) based on the ELISA assays of the saliva samples collected from the control and AD subjects. Units of measure are provided. As can be appreciated from Table 2, there is a significant difference in the biomarker concentrations in the subjects of the healthy and AD groups.

As indicated in Table 2, the AD group subjects had significantly different concentrations, or levels, of the separate biomarker proteins as compared with the biomarker concentrations of the control group of healthy subjects. Levels of IGFBP-2, IGFBP-3, BACE1, TRAIL and IL-6, YKL-40, ICAM-1, VCAM-1, NfL, A1AT, TTR, Neurogranin, and hFABP proteins in the AD group subjects were greater than in the control group whereas GSH biomarker levels in the AD group subjects were reduced as compared with the control subjects with the value p being p=0.05. For example, the mean value of BACE1 in healthy subjects is 567 pg/ml while the BACE1 in AD subjects is a mean value of 932 pg/ml. The biomarker concentrations of the healthy subjects provide a reference value or amount or level of biomarker against which subjects afflicted with AD can be compared.

Salivary IGFBP-2, IGFBP-3, BACE1, GSH, TRAIL, IL-6, YKL-40, ICAM-1 and VCAM-1, NfL, A1AT, TTR, Neurogranin, hFABP protein levels concentrations in the AD group subjects could differentiate those subjects from the control subjects with an AUC value for each biomarker within the range of 0.80-0.96 and a p value of p=0.005.

The specific protein biomarkers BACE1, IGFBP-2, NfL, hFABP, IL-6 and GSH each separately have high diagnostic value in detecting AD and could differentiate the AD subjects from the control subjects with AUC values of 0.95, 0.96, 0.96, 0.92, 0.92 and 0.92 respectively and with a p value of p=0.0050.

Table 2, therefore, shows that the biomarker concentrations among the healthy subjects provide a reference value, or amount, or level of the respective biomarker against which subjects afflicted with AD can be compared. Table 2 further illustrates differences between these reference values provided by the healthy subjects as compared with the biomarker concentrations among the subjects known to be afflicted with AD. The data show that the biomarkers of Table 2 at the exemplary concentrations are indicative of AD in the subject and can be used to predict, detect, monitor, and treat AD in the subject.

Combinations of biomarkers from Table 2 were evaluated to determine their efficacy in the prediction, detection, monitoring, and treatment of AD in a subject and Table 3 contains the data. Table 3 illustrates that representative pairs of salivary biomarkers selected from the biomarkers of Table 2 are powerful and effective tools for evaluating whether individuals may be afflicted with AD. The efficacy of the selected biomarkers is shown by the area under the receiver operating characteristic curve (AUC) values provided in Table 3. The data show that the biomarker combinations are effective to differentiate AD subjects from the healthy control group subjects with a p value of p=0.0050. It is expected that other biomarker combinations selected from the biomarkers presented in Table 2 would also show efficacy in screening subjects for the existence of AD.

TABLE 3
Efficacy of Select Biomarker Combinations in Detecting AD

| | | AUC |
|---|---|---|
| | Biomarker Combinations | Combination |
| 1 | BACE1 and NfL | 0.98 |
| 2 | NfL and IGFBP-2 | 0.98 |
| 3 | BACE1 and IGFBP-2 | 0.97 |
| 4 | NfL and hFABP | 0.97 |
| 5 | NfL and GSH | 0.97 |
| 6 | NfL and IL-6 | 0.97 |
| 7 | BACE1 and hFABP | 0.97 |
| 8 | BACE1 and GSH | 0.97 |
| 9 | BACE1 and IL-6 | 0.96 |
| 10 | IGFBP-2 and hFABP | 0.96 |
| 11 | IGFBP-2 and GSH | 0.96 |
| 12 | IGFBP-2 and IL-6 | 0.96 |

### Example 2

### Efficacy of Salivary Biomarkers in Determination of the Severity of AD

Example 2 was conducted to determine whether the biomarkers of Table 1 are effective to determine the severity of AD in a subject. The study of Example 2 was conducted using 60 matched age and gender healthy control subjects and a further 60 matched age and gender subjects suffering from AD and dementia. Each healthy subject and each AD subject was evaluated for cognitive function by means of MMSE and psychological and additional examinations. The AD subjects were classified by severity of AD also by means of the MMSE protocol. AD subjects were classified as having mild to moderate dementia (MMSE score 11-26) or as having severe dementia (MMSE score 0-10). The clinical and physical examination of the AD subjects was performed by a neurologist.

Saliva samples were taken from each healthy control subject and from each AD subject using the unstimulated drooling method previously described in connection with Example 1. The saliva samples were assayed for the presence and concentration of the salivary biomarkers IGFBP-2, IGFBP-3, BACE1, GSH, TRAIL, IL-6, YKL-40, ICAM-1, VCAM-1, NfL, A1AT, TTR, Neurogranin, and hFABP by means of the ELISA kits and assays described in Example 1.

Table 4 provides the demographics and baseline characteristics of the healthy control subjects and the AD subjects evaluated in Example 2. As shown in Table 4, the mean age of the healthy subjects was 76.9 years (standard deviation SD 1.3), the mean age of the mild to moderate AD subjects was 78.4 years (SD 1.3), and the mean age of the severe AD subjects was 78.1 years (SD 1.5). Subjects were evaluated for salivary folic acid and vitamin B12 levels for the reasons described in Example 1.

TABLE 4
Demographics and Baseline Characteristics of the Subjects

| | | Mild to | | |
|---|---|---|---|---|
| | Healthy | Moderate | Severe | |
| | Control | AD Subjects | AD Subjects | |
| | Subjects | MMSE (11-26) | MMSE (0-10) | P |
| | Mean (SD) | Mean (SD) | Mean (SD) | Value |
| Number of | 60 | 30 | 30 | — |
| subjects | | | | |
| Gender (M:F) | 30:30 | 15:15 | 14:16 | — |
| Age (Years) | 76.9 | 78.4 | 78.1 | 0.86 |
| (SD) | (1.3) | (1.3) | (1.5) | |
| MMSE Mean | 9.4 | 27.3 | 16.9 | <0.005 |
| (SD) | (0.5) | (0.6) | (0.5) | |
| Folic acid | 6.5 | 6.4 | 6.3 | 0.73 |
| (ng/ml) | (1.1) | (1.3) | (1.2) | |
| Mean (SD) | | | | |
| B12 (pg/ml) | 457.5 | 463.4 | 454.6 | 0.66 |
| Mean (SD) | (85.3) | (89.3) | (86.3) | |

TABLE 5
Comparison of Salivary Biomarkers in Control and AD Subjects

| | | | Mild to | | |
|---|---|---|---|---|---|
| | | | Moderate | Severe AD | |
| | | Healthy | AD Subjects | Subjects | |
| | | Control | MMSE | MMSE | |
| | | Subjects | (11-26) | (0-10) | |
| | | Mean (SD) | Mean (SD) | Mean (SD) | P |
| | Biomarkers | Range | Range | Range | Value |
| 1 | IGFBP-2 | 2205(243) | 3206(416) | 3312(503) | <0.005 |
| | (pg/ml) | (1200-2497) | (2500-4019) | 2567-5213) | |
| 2 | IGFBP-3 | 1254(431) | 2409(387) | 2516(412) | <0.005 |
| | (ng/ml) | (439-1700) | (1680-2916) | (2016-3268) | |
| 3 | BACE1 | 568(116) | 940(121) | 1021(256) | <0.005 |
| | (pg/ml) | (167-789) | (700-1267) | (701-1784) | |
| 4 | GSH | 2.1(0.4) | 1.1(0.2) | 0.7(0.4) | <0.005 |
| | (μmol/l) | (1.3-3.1) | (0.7-1.7) | (0.1-1.5) | |
| 5 | TRAIL | 2.3 (1.5) | 4.2(1.1) | 4.6(1.3) | <0.005 |
| | (ng/ml) | (0.5-4.2) | (3.0-6.3) | (3.2-7.9) | |
| 6 | IL-6 | 15.6(3.1) | 29.7(4.2) | 32.7(3.5) | <0.005 |
| | (pg/ml) | (4.6-19.7) | (20.0-38.4) | (27.5-49.6) | |
| 7 | YKL-40 | 24.5(6.8) | 39.5(7.2) | 42.4(6.3) | <0.005 |
| | (ng/ml) | (12.6-32.7) | (30.3-48.6) | (34.2-58.4) | |
| 8 | ICAM-1 | 147(46) | 256(52) | 306(48) | <0.005 |
| | (ng/ml) | (42-198) | (200-354) | (234-482) | |
| 9 | VCAM-1 | 419(112) | 878(124) | 964(131) | <0.005 |
| | (ng/ml) | (216-634) | (450-1247) | (678-1368) | |
| 10 | NfL | 0.3(0.1) | 1.3(0.5) | 1.6(0.3) | <0.005 |
| | (pg/ml) | (0.1-0.4) | (0.3-2.3) | (0.5-3.5) | |
| 11 | A1AT | 1498(406) | 389(125) | 345(102) | <0.005 |
| | (ng/ml) | (783-2453) | (200-648) | (103-589) | |
| 12 | TTR | 19.4(3.6) | 6.1(3.2) | 6.4(2.1) | <0.005 |
| | (μg/ml) | (11.5-24.8) | (2.4-9.8) | (2.0-9.6) | |
| 13 | Neurogranin | 4.1(0.4) | 6.3(0.9) | 6.9(1.3) | <0.005 |
| | (pg/ml) | (1.3-4.8) | (5.0-7.9) | (5.1-9.6) | |
| 14 | hFABP | 0.65(0.14) | 1.30(0.24) | 1.45(0.46) | <0.005 |
| | (ng/ml) | (0.31-0.83) | (0.80-2.10) | (0.89-2.54) | |

Table 5 provides the mean concentration of the biomarkers in the saliva samples for each group, the standard deviation (SD) and the range of the biomarker concentration within the group. Units of measure are provided. As can be appreciated, there is a significant difference in the biomarker concentrations in the subjects of the healthy control group and the subjects of the mild to moderate and severe AD groups. Table 5 demonstrates that the concentration of the separate biomarkers IGFBP-2, IGFBP-3, BACE1, TRAIL, IL-6, YKL-40, ICAM-1, VCAM-1, NfL, Neurogranin, and hFABP were consistently greater in members of the two AD groups of subjects as compared with the healthy, control group of subjects. GSH, A1AT, and TTR concentrations were consistently less. For example, the mean value of BACE1 in healthy subjects is 568 pg/ml while the BACE1 in mild to moderate AD subjects is 940 pg/ml and BACE1 in severe AD subjects is 1021 pg/ml. The biomarker concentrations of the healthy subjects provide a reference value or amount or level of biomarker against which subjects afflicted with AD can be compared. Since the MMSE score of each subject is known, a correlation between the amount of biomarker and the severity of the AD can be determined from Table 5.

Example 2 Table 5 demonstrates that salivary IGFBP-2, IGFBP-3, BACE1, GSH, TRAIL, IL-6, YKL-40, ICAM-1, VCAM-1, NfL, A1AT, TTR, Neurogranin, and hFABP protein concentrations are effective to differentiate subjects with mild to moderate AD from healthy control subjects with a p value of p=0.0050.

Table 5 further demonstrates that the mild to moderate AD subjects (MMSE 11-26) had significant differences in the concentration of IGFBP-2, IGFBP-3, BACE1, GSH, TRAIL, IL-6, YKL-40, ICAM-1, VCAM-1, NfL, A1AT, TTR, Neurogranin, and hFABP as compared with the concentrations of the biomarkers in the severe AD group subjects with a p value of p=0.005.

### Example 3

### Salivary Biomarkers Distinguish Early Stage AD Patients (MCIAD) from Normal Healthy Subjects

Example 3 was undertaken to determine the effectiveness of the biomarkers of Table 1 with respect to differentiating between normal healthy control subjects and subjects known to have very mild levels of AD. The mild AD subjects of Example 3 may be characterized as having “mild cognitive impairment with probable early AD” which is also referred to herein by the acronym MCIAD. The designation MCIAD subjects refers to people who are afflicted with AD but in a very mild form with minimal cognitive impairment. MCIAD subjects are people with a typical MMSE score range of about 26.5 to about 26.8. These MMSE scores position MCIAD subjects in a grouping between normal healthy subjects with no AD affliction and subjects with mild to moderate AD affliction (MMSE scores 11-26) as in Examples 1 and 2.

Use of salivary biomarkers to detect AD in MCIAD subjects is of great importance because the individual may be unaware that he/she is afflicted with AD. Early detection of AD in subjects with no symptoms of AD or with just mild symptoms of AD provides an opportunity for early medical intervention and treatment before the disease progresses further and therefore provides an opportunity for improved healthcare outcomes.

Referring first to Table 6, the data table provides the demographics and baseline characteristics of the subjects of Example 3. As reflected in Table 6, the 48 AD subjects included 28 MCIAD subjects (Mean age 64.5 years, MMSE Range 26.8-26.5, mean MMSE score 26.5) and 20 subjects with more developed AD (Mean age 66.3 years, mean MMSE score 17.2). Twenty normal healthy subjects were selected also as shown in Table 6 (Mean age 64.8, mean MMSE 26.8). All clinical diagnoses of AD in the subjects were done according to the criteria of the Diagnostic and Statistical Manual of Mental Disorders, 4th edition (DSM-IV), the NINCDS-ADRDA and according to the recommendations from the National Institute of Ageing—Alzheimer's Association workgroups on diagnostic guidelines for diagnosis of AD. Albert, M. S., et al., The Diagnosis of Mild Cognitive Impairment Due to Alzheimer's Disease: Recommendations from the National Institute on Aging-Alzheimer's Association Workgroups on Diagnostic Guidelines for Alzheimer's Disease, Alzheimer's Dement. 7:270-279 (2011). The patients were diagnosed by neurologists based on an interview, the MMSE test, and the Clinical Dementia Rating (CDR) test.

Unstimulated saliva samples were taken from each subject as described in Example 1. Assays were performed for the salivary biomarkers IGFBP-2, IGFBP-3, BACE1, GSH, TRAIL, IL-6, YKL-40, ICAM-1, VCAM-1, NfL, A1AT, Neurogranin, and hFABP. The assays were conducted using ELISA kits as described in Example 1. The ELISA assays included determination of the concentration of each biomarker in the saliva specimen.

TABLE 6
Demographics and Baseline Characteristics of the Subjects

| | | Age (years) | Gender | MMSE Score | CDR |
|---|---|---|---|---|---|
| Group | N | Mean (SD) | (F/M) | Mean (SD) | Score |
| Patients with developed | 20 | 66.3(1.5) | 10/10 | 17.2(1.4) | 1.4 |
| Alzheimer's disease (AD) | | | | | |
| Mild cognitive impairment patients | 28 | 64.5(1.4) | 15/13 | 26.5(1.3) | 0.5 |
| with probable early AD (MCIAD) | | | | | |
| MMSE Score Range 26.8-26.5 | | | | | |
| Healthy Control Subjects | 20 | 64.8(1.9) | 10/10 | 26.8(1.6) | 0.5 |

TABLE 7
Comparison of Salivary Biomarkers in Control
and AD Subjects with MCIAD and More Severe AD

| | | Healthy | | | |
|---|---|---|---|---|---|
| | | Control | MCIAD | | |
| | | Subjects | Subjects | AD Subjects | |
| | | Concentration | Concentration | Concentration | |
| | | Mean (SD) | Mean (SD) | Mean (SD) | P |
| | Biomarkers | Range | Range | Range | Value |
| 1 | IGFBP-2 | 2210(230) | 3212(422) | 3403(489) | <0.005 |
| | (pg/ml) | (1590-2400) | (2600-3800) | (2900-4200) | |
| 2 | IGFBP-3 | 1234(420) | 2512(375) | 2578(423) | <0.005 |
| | (ng/ml) | (730-1890) | (2100-3000) | (2106-3267) | |
| 3 | BACE1 | 570(130) | 954(107) | 1341(208) | <0.005 |
| | (pg/ml) | (340-780) | (800-1109) | (1102-1700) | |
| 4 | GSH | 2.2(0.5) | 1.3(0.3) | 0.5(0.3) | <0.005 |
| | (μmol/L) | (1.5-3.2) | (1.0-1.6) | (0.2-0.9) | |
| 5 | TRAIL | 2.1 (1.3) | 4.3(1.1) | 4.7(1.6) | <0.005 |
| | (ng/ml) | (0.6-3.8) | (3.1-5.5) | (3.0-6.8) | |
| 6 | IL-6 | 14.3(2.3) | 30.3(4.5) | 34.8(3.3) | <0.005 |
| | (pg/ml) | (9.2-20.4) | (25-36) | (29.3-42.8) | |
| 7 | YKL-40 | 25.6(6.6) | 41.3(6.3) | 43.6(7.2) | <0.005 |
| | (ng/ml) | (13.5-32.7) | (32-50.2) | (34.4-56.5) | |
| 8 | ICAM-1 | 145(38) | 263(53) | 323(51) | <0.005 |
| | (ng/ml) | (92-204) | (200-320) | (254-438) | |
| 9 | VCAM-1 | 420(114) | 891(142) | 970(123) | <0.005 |
| | (ng/ml) | (230-654) | (720-1065) | (740-1285) | |
| 10 | NfL | 0.1(0.2) | 1.4(0.8) | 1.7(0.4) | <0.005 |
| | (pg/ml) | (0.1-0.4) | (0.5-2.5) | (1.2-2.8) | |
| 11 | A1AT | 1503(443) | 385(118) | 335(105) | <0.005 |
| | (ng/ml) | (1100-2389) | (250-500) | (207-456) | |
| 12 | TTR | 16.3(3.2) | 6.2(3.2) | 6.1(2.3) | <0.005 |
| | (μg/ml) | (9.3-1 | (3.0-10.1) | (2.4-10.7) | |
| 13 | Neurogranin | 3.9(0.5) | 6.4(0.5) | 7.1(1.1) | <0.005 |
| | (pg/ml) | (2.8-4.3) | (4.5-7.0) | (5.6-9.3) | |
| 14 | hFABP | 0.63(0.12) | 1.31(0.22) | 1.47(0.41) | <0.005 |
| | (ng/ml) | (0.34-0.89) | (0.96-1.78) | (1.05-2.45) | |

The data of Table 7 provide the mean concentration of the biomarkers in the saliva samples of each group, the standard deviation (SD), and the biomarker concentration range for each subject evaluated grouped by severity of AD. Units of measure are provided. As can be appreciated, there is a significant difference in the biomarker concentrations in the subjects of the healthy control group and the subjects of the MCIAD group. Further, there is a significant difference between the MCIAD group subjects and the subjects with more developed AD. Table 7 demonstrates that the concentration of the separate biomarkers IGFBP-2, IGFBP-3, BACE1, TRAIL, IL-6, YKL-40, ICAM-1, VCAM-1, NfL, Neurogranin, and hFABP were consistently greater in members of the two AD groups of subjects as compared with the healthy, control group of subjects. GSH, A1AT, and TTR concentrations were consistently less. For example, the mean value of hFABP in healthy subjects is 0.63 ng/ml while the hFABP in MCIAD subjects is 1.31 ng/ml and hFABP in the group of more developed AD subjects is 1.47 ng/ml.

The biomarker concentrations of the healthy subjects provide a reference value or amount or level of biomarker against which subjects afflicted with AD can be compared. Since the MMSE score of each subject is known, a correlation between the amount of biomarker and the severity of the AD can be determined from Table 7. This strong correlation (p value greater than 0.0050) is particularly important when seeking to differentiate healthy subjects from MCIAD subjects because of the lack of outward manifestations of differences between subjects in these two groups.

Table 7 further demonstrates that the MCIAD subjects (MMSE range 26.8-26.5) had significant differences in the concentration of IGFBP-2, IGFBP-3, BACE1, GSH, TRAIL, IL-6, YKL-40, ICAM-1, VCAM-1, NfL, A1AT, TTR, Neurogranin, and hFABP as compared with the concentrations of the biomarkers in the AD group subjects with a p value greater than 0.005.

Therefore, Example 3 demonstrates that the salivary biomarkers IGFBP-2, IGFBP-3, BACE1, TRAIL, IL-6, YKL-40, ICAM-1, VCAM-1, NfL, Neurogranin, and hFABP provide excellent discrimination between normal healthy subjects and MCIAD subjects and between MCIAD subjects and subjects with more severe AD. The data of Example 3 demonstrate that the biomarkers correlate highly with very subtle differences in AD severity within the subjects. Discrimination between normal healthy subjects and MCIAD subjects, in particular, is important to identify early-stage AD in subjects with few, if any, outward symptoms of the disease.

Also in Example 3, an analysis was conducted to determine whether there was any correlation between age of the subject and detection of the biomarkers. As indicated by the data of Table 8, no correlations were found between age and biomarkers. Accordingly, the biomarkers of Tables 1 and 8 can be used effectively for screening, diagnosis, detection, monitoring, or prognosis for AD irrespective of age of the subject.

TABLE 8
Correlation Between Salivary Biomarker with Age

| | Biomarkers | R | P Value |
|---|---|---|---|
| 1 | IGFBP2 | 0.05 | 0.73 |
| 2 | IGFBP-3 | 0.09 | 0.74 |
| 3 | BACE1 | 0.07 | 0.91 |
| 4 | GSH | −0.04 | 0.89 |
| 5 | TRAIL | 0.08 | 0.67 |
| 6 | IL-6 | 0.07 | 0.74 |
| 7 | YKL-40 | 0.04 | 0.52 |
| 8 | ICAM-1 | 0.08 | 0.46 |
| 9 | VCAM-1 | 0.06 | 0.72 |
| 10 | NfL | 0.02 | 0.63 |
| 11 | A1AT | −0.13 | 0.81 |
| 12 | TTR | −0.06 | 0.83 |
| 13 | Neurogranin | 0.07 | 0.65 |
| 14 | hFABP | 0.09 | 0.98 |

### Example 4

### Efficacy of Multiple Biomarker Combinations in Detection of AD

Example 4 demonstrates that further combinations of biomarkers are efficacious with respect to detection of AD and for the prediction, detection, monitoring, treatment, management and general diagnosis and prognosis of AD in a subject. The combinations may be implemented as part of a biomarker panel on a solid support used, for example, in an ELISA type assay. Example 4 was conducted to analyze the accuracy of a biomarker panel based on combinations of IGFBP-2, IGFBP-3, BACE1, GSH, TRAIL, IL-6, YKL-40, ICAM-1, VCAM-1, NfL, A1AT, TTR, Neurogranin, and hFABP for the diagnosis of, and discrimination between, AD and control subjects. Combinations of these biomarkers were evaluated as indicated in Tables 9A and 9B.

According to Example 4, a statistical comparison of the control and symptomatic subjects of Examples 1 and 2 (by the combination of the salivary biomarkers in Examples 1 and 2) was performed using the two-tailed t-test using GraphPad Prism for Windows, v. 5.01 (GraphPad Software, San Diego, Calif.). Receiver operating characteristic curves (ROC) were generated using the R software environment for statistical computing and graphics (R Foundation for Statistical Computing, Vienna, Austria). Also in Example 4, a statistical comparison of the control and asymptomatic subjects of Example 3 was performed using the two-tailed t-test in the same manner as with the Example 1 and 2 subjects.

Tables 9A and 9B which follow provide an ROC analysis and diagnostic performance for various combinations of fourteen salivary biomarker combinations, namely, IGFBP-2 (A), IGFBP-3 (B), BACE1 (C), GSH (D), TRAIL (E), IL-6 (F), YKL-40 (G), ICAM-1 (H), VCAM-1 (I), NfL (J), A1AT (K), TTR (L), Neurogranin (M), and hFABP (N) for the diagnosis of and discrimination between the control subjects and subjects with AD from Examples 1 and 2.

TABLE 9A
ROC Analysis of Salivary Biomarker Combinations
in Detection of AD—Example 1 and 2 Subjects

| | Biomarker Combinations | | | | | | |
|---|---|---|---|---|---|---|---|
| | A | AB | ABC | ABCD | ABCDE | ABCDEF | ABCDEFG |
| AUC | 0.97 | 0.98 | 0.98 | 0.98 | 0.98 | 0.98 | 0.98 |
| Sensitivity | 0.86 | 0.87 | 0.89 | 0.91 | 0.92 | 0.95 | 0.96 |
| Specificity | 0.85 | 0.86 | 0.88 | 0.89 | 0.91 | 0.93 | 0.95 |

TABLE 9B
ROC Analysis of Salivary Biomarker Combinations
in Detection of AD—Example 1 and 2 Subjects

| | Biomarker Combinations | | | | | | |
|---|---|---|---|---|---|---|---|
| | ABCDEFGH | ABCDEFGHI | A to J | A to K | A to L | A to M | A to N |
| AUC | 0.98 | 0.99 | 0.99 | 0.99 | 0.99 | 0.99 | 0.99 |
| Sensitivity | 0.97 | 0.98 | 0.98 | 0.98 | 0.98 | 0.98 | 0.99 |
| Specificity | 0.96 | 0.98 | 0.98 | 0.98 | 0.98 | 0.98 | 0.99 |

As shown in Tables 9A and 9B, the ROC analysis established diagnostic sensitivity and specificity for AD using panels of many different biomarker combinations. The salivary biomarker combination identified in Tables 9A and 9B of IGFBP-2 (A), IGFBP-3 (B), BACE1 (C), GSH (D), TRAIL (E), IL-6 (F), YKL-40 (G), ICAM-1 (H), VCAM-1 (I), NfL (J), A1AT (K), TTR (L), Neurogranin (M), hFABP (N) have high diagnostic values for diagnosis of AD as compared to other models, i.e., individual biomarker types only (e.g., Table 9A, combination 1). Accordingly, it can be expected that the combination of any two or more of the biomarkers in Tables 9A and 9B would have high diagnostic values for screening, monitoring, diagnosis, and prognosis of AD.

Results predictive of AD in a subject are increased further for combinations of panels including two or more biomarkers in combination as indicated by combinations 2-14 in Tables 9A and 9B which have particularly predictive AUC, sensitivity and specificity values indicative of AD in a subject. The efficacy of biomarker combinations selected from Tables 9A and 9B in detecting AD is further confirmed by the data of Example 1, Table 3 in which it is demonstrated that combinations including the salivary biomarkers BACE1, IGFBP-2, NfL, hFABP, IL-6, and GSH were respectively demonstrated to be effective in detecting AD in subjects.

The combination model including IGFBP-2, IGFBP-3, and BACE1 (combination ABC) demonstrates excellent diagnostic values for diagnosis of AD and is an especially efficacious model given that AD can be detected with a high level of confidence with just three biomarkers. Use of relatively fewer biomarkers (e.g., three biomarkers) is desirable for cost reduction and simplicity purposes.

Another excellent combination model according to Table 9A includes IGFBP-2, IGFBP-3, BACE1, GSH, TRAIL, and IL-6. This six biomarker combination also has high diagnostic values for diagnosis of AD. The data demonstrate that the biomarkers of Tables 9A and 9B correlate strongly with AD in subjects symptomatic for AD.

Tables 9C and 9D show efficacy of biomarkers in detecting AD in the asymptomatic subjects of Example 3. Tables 9C and 9D provide a further ROC analysis and diagnostic performance analysis based on the asymptomatic subjects of Example 3 and employing salivary biomarker combinations different from those of Tables 9A and 9B. Tables 9C and 9D demonstrate that IGFBP-2 (A) and Neurogranin (M) in paired combination are efficacious for the diagnosis of, and discrimination between, asymptomatic subjects with AD and healthy control subjects. This pair demonstrates that embodiments of the invention may include biomarker pairs other than those identified in Table 3. Addition of further biomarkers may be implemented for improved diagnosis of, and discrimination between, asymptomatic subjects with AD and healthy control subjects with a high degree of certainty as indicated in Tables 9C and 9D.

TABLE 9C
ROC Analysis of Salivary Biomarker Combinations
in Detection of Asymptomatic AD—Example 3 Subjects

| | Biomarker Combinations | | | | | | |
|---|---|---|---|---|---|---|---|
| | A | AM | AMD | AMCD | AMBCDE | AMBCDEF | A to G |
| AUC | 0.97 | 0.99 | 0.99 | 0.99 | 0.99 | 0.99 | 0.99 |
| Sensitivity | 0.86 | 0.90 | 0.96 | 0.98 | 0.98 | 0.98 | 0.98 |
| Specificity | 0.85 | 0.90 | 0.96 | 0.98 | 0.98 | 0.98 | 0.99 |

TABLE 9D
ROC Analysis of Salivary Biomarker Combinations
in Detection of Asymptomatic AD—Example 3 Subjects

| | Biomarker Combinations | | | | | |
|---|---|---|---|---|---|---|
| | A to H | A to I | A to J | A to K | A to L | A to N |
| AUC | 0.99 | 0.99 | 0.99 | 0.99 | 0.99 | 0.99 |
| Sensitivity | 0.99 | 0.99 | 0.99 | 0.99 | 0.99 | 0.99 |
| Specificity | 0.99 | 0.99 | 0.99 | 0.99 | 0.99 | 0.99 |

The efficacy of biomarker combinations selected from Tables 9C and 9D in detecting asymptomatic AD are again consistent with the data of Example 1, Table 3 where combinations including BACE1, IGFBP-2, NfL, hFABP, IL-6, and GSH were respectively demonstrated to have high predictive value in detecting AD in individual asymptomatic subjects and groups of asymptomatic subjects.

### Example 5

### Reproducibility of Results

Example 5 was conducted to evaluate the reproducibility and stability of salivary biomarkers. Reproducibility of results is important, for example, to confirm that examples of salivary biomarkers can be used to reliably monitor the progression of AD in a subject over a period of time. A salivary biomarker sample with reproducible usage may provide a baseline by which to measure a subject's improvement or deterioration.

According to Example 5, saliva samples from ten healthy subjects and separately ten AD subjects from Example 1 were obtained. The samples were randomly arranged and labeled such that the laboratory could not identify the subjects sampled.

For each analysis, the assay reproducibility of blinded quality control replicates was examined using the coefficient of variation (CV), a commonly used statistical analysis technique to describe laboratory technical error, and a determination was made of the effect of delayed sample processing on analyte concentrations in frozen samples at −80° C. (at twenty four hours, seven days and fourteen days after sampling, i.e. reproducibility with delayed processing). Reproducibility was assessed over a one-week and two-week period for salivary biomarkers, by taking samples at seven days and fourteen days. The CV was determined by estimating the SD (standard deviation) of the quality control values, divided by the mean of these values, multiplied by 100. Inter-observer and intra-observer variances were estimated from repeated sample measurements using a random effects model, with sample identification number as the random variable.

To assess reproducibility, the ICC (Intraclass Correlation Coefficient) values were calculated by dividing the intra-observer variance by the sum of the within- and inter-observer variances. Ninety-five percent (95%) confidence intervals (CI) were also calculated. The inter- and intra-observer CVs were determined by taking the square root of the inter- and intra-observer variance components from the random effects mixed model on the In [log] transformed scale, with approximate estimates derived by the eta method. Rosner, B., Fundamentals of Biostatistics. Duxbury (2006). An ICC of <0.40 indicates poor reproducibility, an ICC of 0.40 to 0.8 indicates fair to good reproducibility, and an ICC of more than 0.8 indicates excellent reproducibility. Results are shown in Tables 10 and 11.

Table 10 provides ICCs calculated for delayed analysis and processing of a single frozen sample at day one, day seven, and day fourteen for salivary biomarkers in subjects. Tables 10-11 provide ICCs calculated of samples tested at various time points (day one, day seven and day fourteen) in all subjects.

TABLE 10
Intraclass Correlation Coefficient—Single Saliva Sample in Subjects

| | | Number of | Intra-observer | | | Inter-observer | | | | | |
|---|---|---|---|---|---|---|---|---|---|---|---|
| | | participants/ | CV (%) | | | CV (%) | | | ICC (95% CIs) | | |
| | | number of | Day | Day | Day | Day | Day | Day | Day | Day | Day |
| Biomarker | | time points | 1 | 7 | 14 | 1 | 7 | 14 | 1 | 7 | 14 |
| 1 | IGFBP-2 | 20/3 | 1.2 | 1.2 | 1.3 | 2.2 | 2.1 | 3.0 | 0.92 | 0.90 | 0.93 |
| 2 | IGFBP-3 | 20/3 | 1.4 | 1.3 | 1.4 | 2.1 | 2.2 | 2.8 | 0.93 | 0.94 | 0.93 |
| 3 | BACE1 | 20/3 | 1.2 | 1.4 | 1.4 | 2.2 | 2.1 | 2.9 | 0.94 | 0.94 | 0.92 |
| 4 | GSH | 20/3 | 1.3 | 1.5 | 1.5 | 2.4 | 2.3 | 3.1 | 0.91 | 0.92 | 0.94 |
| 5 | TRAIL | 20/3 | 1.2 | 1.2 | 1.2 | 3.1 | 2.8 | 2.9 | 0.94 | 0.93 | 0.92 |
| 6 | IL-6 | 20/3 | 1.3 | 1.2 | 1.1 | 2.3 | 2.3 | 2.4 | 0.91 | 0.92 | 0.93 |
| 7 | YKL-40 | 20/3 | 1.2 | 1.4 | 1.4 | 2.1 | 2.1 | 3.2 | 0.92 | 0.93 | 0.92 |
| 8 | ICAM-1 | 20/3 | 1.3 | 1.3 | 1.3 | 2.4 | 2.8 | 3.1 | 0.93 | 0.91 | 0.93 |
| 9 | VCAM-1 | 20/3 | 1.4 | 1.5 | 1.4 | 2.2 | 2.5 | 3.1 | 0.92 | 0.94 | 0.93 |
| 10 | NfL | 20/3 | 1.3 | 1.4 | 1.5 | 2.4 | 2.5 | 3.0 | 0.91 | 0.92 | 0.91 |
| 11 | A1AT | 20/3 | 1.4 | 1.3 | 1.3 | 2.8 | 2.8 | 3.1 | 0.93 | 0.92 | 0.90 |
| 12 | TTR | 20/3 | 1.4 | 1.3 | 1.2 | 2.1 | 2.2 | 3.0 | 0.93 | 0.94 | 0.94 |
| 13 | Neurogranin | 20/3 | 1.4 | 1.3 | 1.4 | 2.4 | 2.9 | 3.1 | 0.92 | 0.92 | 0.91 |
| 14 | hFABP | 20/3 | 1.1 | 1.4 | 1.3 | 2.3 | 2.1 | 2.5 | 0.92 | 0.93 | 0.93 |

TABLE 11
Intraclass Correlation Coefficient Time Point Testing in All Subjects

| | | Number of | | | | | | | | | |
|---|---|---|---|---|---|---|---|---|---|---|---|
| | | participants/ | Intra-observer | | | Inter-observer | | | | | |
| | | number of | CV (%) | | | CV (%) | | | ICC (95% CIs) | | |
| Biomarker | | time points | Day 1 | Day 7 | Day 14 | Day 1 | Day 7 | Day 14 | Day 1 | Day 7 | Day 14 |
| 1 | IGFBP2 | 20/3 | 1.3 | 1.5 | 1.5 | 2.1 | 2.6 | 2.9 | 0.9 | 0.93 | 0.92 |
| 2 | IGFBP-3 | 20/3 | 1.4 | 1.3 | 1.4 | 2.1 | 2.5 | 2.6 | 0.93 | 0.94 | 0.93 |
| 3 | BACE1 | 20/3 | 1.1 | 1.3 | 1.7 | 2.3 | 2.6 | 2.7 | 0.92 | 0.93 | 0.92 |
| 4 | GSH | 20/3 | 1.2 | 1.2 | 1.3 | 1.9 | 2.4 | 2.5 | 0.91 | 0.92 | 0.91 |
| 5 | TRAIL | 20/3 | 1.4 | 1.3 | 1.6 | 2.4 | 2.8 | 2.6 | 0.90 | 0.91 | 0.91 |
| 6 | IL-6 | 20/3 | 1.2 | 1.4 | 1.5 | 2.4 | 2.4 | 2.4 | 0.94 | 0.91 | 0.93 |
| 7 | YKL-40 | 20/3 | 1.2 | 1.4 | 1.4 | 2.2 | 2.8 | 2.8 | 0.94 | 0.94 | 0.90 |
| 8 | ICAM-1 | 20/3 | 1.1 | 1.3 | 1.2 | 2.0 | 2.6 | 2.3 | 0.92 | 0.93 | 0.93 |
| 9 | VCAM-1 | 20/3 | 1.1 | 1.4 | 1.5 | 2.1 | 2.7 | 2.9 | 0.93 | 0.94 | 0.94 |
| 10 | NfL | 20/3 | 1.3 | 1.4 | 1.9 | 2.3 | 2.4 | 2.8 | 0.92 | 0.93 | 0.93 |
| 11 | A1AT | 20/3 | 1.1 | 1.4 | 1.8 | 2.1 | 2.5 | 2.5 | 0.94 | 0.94 | 0.96 |
| 12 | TTR | 20/3 | 1.3 | 1.5 | 1.4 | 2.2 | 2.3 | 2.6 | 0.93 | 0.92 | 0.95 |
| 13 | Neurogranin | 20/3 | 1.2 | 1.3 | 1.6 | 2.2 | 2.4 | 2.6 | 0.93 | 0.91 | 0.92 |
| 14 | hFABP | 20/3 | 1.3 | 1.2 | 1.4 | 2.3 | 2.5 | 2.8 | 0.92 | 0.93 | 0.90 |

The data of Example 5 demonstrate that the ICCs for the range of salivary biomarkers were high (ICCs of 0.9-0.95), indicating good to excellent reproducibility and stability. Example 5 demonstrates that the biomarkers of the study are stable and easy to reproduce.

Those skilled in the art will recognize that numerous modifications and changes may be made to the preferred embodiments without departing from the scope of the claimed invention. It will, of course, be understood that modifications of the invention, in its various aspects, will be apparent to those skilled in the art. Other embodiments are possible, their specific designs depending upon the particular application. As such, the scope of the invention should not be limited by the particular embodiments herein described, but should be defined only by the appended claims and equivalents thereof.

1. A method of detecting biomarkers indicative of Alzheimer's disease (AD) in a subject comprising:
(a) obtaining a saliva sample from the subject; and
(b) detecting whether one or more biomarkers selected from the group consisting of Insulin-like growth factor binding protein-2 (IGFBP-2), Insulin-like growth factor binding protein-3 (IGFBP-3), Beta-secretase 1 (BACE1), Reduced glutathione (GSH), TNF-related apoptosis-including ligand (TRAIL), Chitinase-3-like protein 1 (YKL-40), Intercellular Adhesion Molecule 1 (ICAM-1), Neurofilament protein L (NfL), Alpha-1 antitrypsin (A1AT), Transthyretin (TTR), Neurogranin, and Human heart fatty acid binding protein (hFABP), is or are present in the saliva sample by contacting the saliva sample with binding agents capable of binding with a specific one of the biomarkers and detecting the binding between the binding agents and the biomarker specific thereto.

2. The method of claim 1 wherein the group of one or more biomarkers further includes Interleukin 6 (IL-6) and Vascular cell adhesion protein 1 (VCAM-1) and wherein the detecting further includes detecting that the one or more detected biomarker is present in a reference value or amount for the respective biomarker comprising: IGFBP-2 above about 2500 pg/ml, IGFBP-3 above about 1680 ng/ml, BACE1 above about 700 pg/ml, GSH less than about 1.7 μmol/l, TRAIL above about 3 ng/ml, IL-6 above about 20 pg/ml, YKL-40 above about 30 ng/ml, ICAM-1 above about 200 ng/ml, VCAM-1 above about 450 ng/ml, NfL above about 0.3 pg/ml, A1AT less than about 650 ng/ml, TTR less than about 10 μg/ml, Neurogranin above about 5 pg/ml, and hFABP above about 0.80 ng/ml,
wherein detection that the biomarker is present in the reference value or amount is indicative that the subject is afflicted with AD.

3. The method of claim 2 wherein the subject is being evaluated for severe AD and the detecting further includes detecting that the one or more detected biomarker is present in a reference value or amount for the respective biomarker comprising: IGFBP-2 in the range of about 2567 pg/ml to about 5213 pg/ml, IGFBP-3 in the range of about 2016 ng/ml to about 3268 ng/ml, BACE1 in the range of about 701 pg/ml to about 1784 pg/ml, GSH in the range of about 0.1 mol/1 to about 1.5 μmol/l, TRAIL in the range of about 3.2 ng/ml to about 7.9 ng/ml, IL-6 in the range of about 27.5 pg/ml to about 49.6 pg/ml, YKL-40 in the range of about 34.2 ng/ml to about 58.4 ng/ml, ICAM-1 in the range of about 234 ng/ml to about 482 ng/ml, VCAM-1 in the range of about 678 ng/ml to about 1368 ng/ml, NfL in the range of about 0.5 pg/ml to about 3.5 pg/ml, A1AT in the range of about 103 ng/ml to about 589 ng/ml, TTR in the range of about 2.0 μg/ml to about 9.6 μg/ml, Neurogranin in the range of about 5.1 pg/ml to about 9.6 pg/ml and hFABP in the range of about 0.89 ng/ml to about 2.54 ng/ml,
wherein detection that the biomarker is present in the reference value or amount is indicative that the subject is afflicted with severe AD equivalent to an MMSE score of about 0 to about 10.

4. The method of claim 2 wherein the subject is being evaluated for mild to moderate AD and the detecting further includes detecting that the one or more detected biomarker is present in a reference value or amount for the respective biomarker comprising: IGFBP-2 in the range of about 2500 pg/ml to about 4019 pg/ml, IGFBP-3 in the range of about 1680 ng/ml to about 2916 ng/ml, BACE1 in the range of about 700 pg/ml to about 1267 pg/ml, GSH in the range of about 0.7 μmol/l to about 1.7 μmol/l, TRAIL in the range of about 3.0 ng/ml to about 6.3 ng/ml, IL-6 in the range of about 20.0 pg/ml to about 38.4 pg/ml, YKL-40 in the range of about 30.3 ng/ml to about 48.6 ng/ml, ICAM-1 in the range of about 200 mg/ml to about 354 ng/ml, VCAM-1 in the range of about 450 mg/ml to about 1247 ng/ml, NfL in the range of about 0.3 pg/ml to about 2.3 pg/ml, A1AT in the range of about 200 ng/ml to about 648 ng/ml, TTR in the range of about 2.4 μg/ml to about 9.8 μg/ml, Neurogranin in the range of about 5.0 pg/ml to about 7.9 pg/ml, and hFABP in the range of about 0.80 ng/ml to about 2.10 ng/ml,
wherein detection that the biomarker is present in the reference value or amount is indicative that the subject is afflicted with mild to moderate AD equivalent to an MMSE score of about 11 to about 26.

5. The method of claim 2 wherein the human subject is being evaluated for AD yet is asymptomatic of AD and the detecting further includes detecting that the one or more detected biomarker is present in a reference value or amount for the respective biomarker comprising: IGFBP-2 in the range of about 2600 pg/ml to about 3800 pg/ml, IGFBP-3 in the range of about 2100 ng/ml to about 3000 ng/ml, BACE1 in the range of about 800 pg/ml to about 1110 pg/ml, GSH in the range of about 1 μmol/l to about 1.6 μmol/l, TRAIL in the range of about 3 ng/ml to about 5.5 ng/ml, IL-6 in the range of about 25 pg/ml to about 36 pg/ml, YKL-40 in the range of about 32 ng/ml to about 50 ng/ml, ICAM-1 in the range of about 200 ng/ml to about 320 ng/ml, VCAM-1 in the range of about 720 ng/ml to about 1065 ng/ml, NfL in the range of about 0.5 pg/ml to about 2.5 pg/ml, A1AT in the range of about 250 ng/ml to about 500 ng/ml, TTR in the range of about 3 μg/ml to about 10 μg/ml, Neurogranin in the range of about 4.5 pg/ml to about 7 pg/ml, and hFABP in the range of about 0.96 ng/ml to about 1.78 ng/ml,
wherein detection that the biomarker is present in the reference value or amount range is indicative that the subject is afflicted with mild cognitive impairment with probable early AD (MCIAD) equivalent to an MMSE score of about 26.5 to about 26.8.

6. The method of claim 5 wherein the subject is a human subject.

7. The method of claim 5 further comprising, before or after obtaining the saliva sample, determining that the subject is afflicted with AD by means other than the biomarkers.

8. The method of claim 2 wherein detecting further includes detecting the one or more biomarkers using an assay selected from the group of assays consisting of a lateral flow immunochromatographic assay (LFA), an enzyme-linked immunosorbent assay (ELISA), an enzyme-linked fluorescence polarization immunoassay (FPIA), a homogeneous immunoassay, a quantitative point-of-care assay using determination of chemiluminescence, fluorescence, magnetic particles, or latex agglutination, a gel electrophoresis assay, a gas chromatograph-mass spectrometry (GC-MS) assay, a separation immunoassay, a heterogeneous immunoassay, a homogenous immunoassay, a latex agglutination assay, a western blot assay, and a biosensor assay.

9. The method of claim 8 wherein the binding agents are selected from the group consisting of antibodies, antigens, nanoparticles, aptamers, inhibitors, substrates, cofactors, coenzymes, lectins, nucleic acids, protein A, protein G, nonbiological ligands, boronates, triazine dyes, and metal-ion chelates.

10. The method of claim 9 wherein the binding agents are secured to a solid support.

11. The method of claim 2 wherein the binding agents are selected for their capability to bind with a specific one of the biomarkers in the combination of biomarkers selected from the group consisting of
(1) BACE1 and NfL;
(2) NfL and IGFBP-2;
(3) BACE1 and IGFBP-2;
(4) NfL and hFABP;
(5) NfL and GSH;
(6) NfL and Interleukin 6 (IL-6);
(7) BACE1 and hFABP;
(8) BACE1 and GSH;
(9) BACE1 and IL-6;
(10) IGFBP-2 and hFABP;
(11) IGFBP-2 and GSH;
(12) IGFBP-2 and IL-6;
(13) IGFBP-2 and IGFBP-3; and
(14) IGFBP-2 and Neurogranin.

12. The method of claim 11 wherein the combinations of the biomarkers increase the specificity and sensitivity for detection of AD from about 86 to 99%.

13. The method of claim 11 wherein the binding agents further include binding agents capable of binding with a specific one of the biomarkers IGFBP-2, IGFBP-3, and BACE1.

14. The method of claim 13 wherein the binding agents further include binding agents capable of binding with the biomarker GSH.

15. The method of claim 14 wherein the binding agents further include binding agents capable of binding with the biomarker TRAIL.

16. The method of claim 15 wherein the binding agents further include binding agents capable of binding with the biomarker IL-6.

17. The method of claim 16 wherein the binding agents further include binding agents capable of binding with the biomarker YKL-40.

18. The method of claim 17 wherein the binding agents further include binding agents capable of binding with the biomarker ICAM-1.

19. The method of claim 18 wherein the binding agents further include binding agents capable of binding with the biomarker VCAM-1.

20. The method of claim 19 wherein the binding agents further include binding agents capable of binding with the biomarker NfL.

21. The method of claim 20 wherein the binding agents further include binding agents capable of binding with the biomarker A1AT.

22. The method of claim 21 wherein the binding agents further include binding agents capable of binding with the biomarker TTR.

23. The method of claim 22 wherein the binding agents further include binding agents capable of binding with the biomarker Neurogranin.

24. The method of claim 23 wherein the binding agents further include binding agents capable of binding with the biomarker hFABP.

25. The method of claim 11 wherein the binding agents further include binding agents capable of binding with a specific one of the biomarkers IGFBP-2, Neurogranin, and GSH.

26. The method of claim 25 wherein the binding agents further include binding agents capable of binding with the biomarker BACE1.

27. The method of claim 26 wherein the binding agents further include binding agents capable of binding with the biomarker TRAIL.

28. The method of claim 27 wherein the binding agents further include binding agents capable of binding with the biomarker IL-6.

29. The method of claim 28 wherein the binding agents further include binding agents capable of binding with the biomarker YKL-40.

30. The method of claim 29 wherein the binding agents further include binding agents capable of binding with the biomarker ICAM-1.

31. The method of claim 30 wherein the binding agents further include binding agents capable of binding with the biomarker VCAM-1.

32. The method of claim 31 wherein the binding agents further include binding agents capable of binding with the biomarker NfL.

33. The method of claim 32 wherein the binding agents further include binding agents capable of binding with the biomarker A1AT.

34. The method of claim 33 wherein the binding agents further include binding agents capable of binding with the biomarker TTR.

35. The method of claim 34 wherein the binding agents further include binding agents capable of binding with the biomarkers Neurogranin and hFABP.

36. The method of claim 5 further comprising diagnosing the subject as having AD if the at least one biomarker in the saliva sample meets the reference value or amount.

37. The method of claim 36 further including:
(c) administering a drug or agent to the subject; and
(d) comparing changes in the reference value or amount of at least one of said biomarkers over time responsive to the drug or agent to determine whether the subject has benefited from treatment with said drug or agent.

38. A system for detecting salivary biomarkers indicative of Alzheimer's disease (AD) in a saliva sample obtained from a subject to determine whether the subject is afflicted with AD or the severity of AD in the subject, the system comprising:
(a) binding agents specific to one or more salivary biomarker selected from the group consisting of Insulin-like growth factor binding protein-2 (IGFBP-2), Insulin-like growth factor binding protein-3 (IGFBP-3), Beta-secretase 1 (BACE1), Reduced glutathione (GSH), TNF-related apoptosis-including ligand (TRAIL), Chitinase-3-like protein 1 (YKL-40), Intercellular Adhesion Molecule 1 (ICAM-1), Neurofilament protein L (NfL), Alpha-1 antitrypsin (A1AT), Transthyretin (TTR), Neurogranin, and Human heart fatty acid binding protein (hFABP), and combinations thereof;
(b) a measurable label associated with the binding agents that indicates a proportional reaction based on the amount of biomarker present in the saliva sample; and
(c) a measurement device operable to utilize the label to provide a qualitative, semi-quantitative, or quantitative measure of the one or more salivary biomarker indicative of whether the subject is afflicted with AD or the severity of AD in the subject.

39. The system of claim 38 wherein the group of at least one binding agent further includes binding agents specific to Interleukin 6 (IL-6) and Vascular cell adhesion protein 1 (VCAM-1) and wherein the measurement device is operable to detect that the one or more detected biomarker is present in a reference value or amount for the respective biomarker comprising: IGFBP-2 above about 2500 pg/ml, IGFBP-3 above about 1680 ng/ml, BACE1 above about 700 pg/ml, GSH less than about 1.7 μmol/l, TRAIL above about 3 ng/ml, IL-6 above about 20 pg/ml, YKL-40 above about 30 ng/ml, ICAM-1 above 200 ng/ml, VCAM-1 above about 450 ng/ml, NfL above about 0.3 pg/ml, A1AT less than about 650 ng/ml, TTR less than about 10 μg/ml, Neurogranin above about 5 pg/ml, and hFABP above about 0.80 ng/ml,
wherein measurement that the biomarker is present in the reference value or amount is indicative that the subject is afflicted with AD.

40. The system of claim 39 wherein the measurement device is operable to detect that the one or more detected biomarker is present in a reference value or amount for the respective biomarker comprising: IGFBP-2 in the range of about 2567 pg/ml to about 5213 pg/ml, IGFBP-3 in the range of about 2016 ng/ml to about 3268 ng/ml, BACE1 in the range of about 701 pg/ml to about 1784 pg/ml, GSH in the range of about 0.1 mol/1 to about 1.5 μmol/l, TRAIL in the range of about 3.2 ng/ml to about 7.9 ng/ml, IL-6 in the range of about 27.5 pg/ml to about 49.6 pg/ml, YKL-40 in the range of about 34.2 ng/ml to about 58.4 ng/ml, ICAM-1 in the range of about 234 ng/ml to about 482 ng/ml, VCAM-1 in the range of about 678 ng/ml to about 1368 ng/ml, NfL in the range of about 0.5 pg/ml to about 3.5 pg/ml, A1AT in the range of about 103 ng/ml to about 589 ng/ml, TTR in the range of about 2.0 μg/ml to about 9.6 μg/ml, Neurogranin in the range of about 5.1 pg/ml to about 9.6 pg/ml and hFABP in the range of about 0.89 ng/ml to about 2.54 ng/ml, and
wherein detection that the biomarker is present in the reference value or amount is indicative that the subject is afflicted with severe AD equivalent to an MMSE score of about 0 to about 10.

41. The system of claim 39 wherein the measurement device is operable to detect that the one or more detected biomarker is present in a reference value or amount for the respective biomarker comprising: IGFBP-2 in the range of about 2500 pg/ml to about 4019 pg/ml, IGFBP-3 in the range of about 1680 ng/ml to about 2916 ng/ml, BACE1 in the range of about 700 pg/ml to about 1267 pg/ml, GSH in the range of about 0.7 μmol/l to about 1.7 μmol/l, TRAIL in the range of about 3.0 ng/ml to about 6.3 ng/ml, IL-6 in the range of about 20.0 pg/ml to about 38.4 pg/ml, YKL-40 in the range of about 30.3 ng/ml to about 48.6 ng/ml, ICAM-1 in the range of about 200 mg/ml to about 354 ng/ml, VCAM-1 in the range of about 450 mg/ml to about 1247 ng/ml, NfL in the range of about 0.3 pg/ml to about 2.3 pg/ml, A1AT in the range of about 200 ng/ml to about 648 ng/ml, TTR in the range of about 2.4 μg/ml to about 9.8 μg/ml, Neurogranin in the range of about 5.0 pg/ml to about 7.9 pg/ml, and hFABP in the range of about 0.80 ng/ml to about 2.10 ng/ml,
wherein detection that the biomarker is present in the reference value or amount is indicative that the subject is afflicted with mild to moderate AD equivalent to an MMSE score of about 11 to about 26.

42. The system of claim 39 wherein the measurement device is operable to detect that the one or more detected biomarker is present in a reference value or amount for the respective biomarker comprising: IGFBP-2 in the range of about 2600 pg/ml to about 3800 pg/ml, IGFBP-3 in the range of about 2100 ng/ml to about 3000 ng/ml, BACE1 in the range of about 800 pg/ml to about 1100 pg/ml, GSH in the range of about 1 μmol/l to about 1.6 μmol/l, TRAIL in the range of about 3 ng/ml to about 5.5 ng/ml, IL-6 in the range of about 25 pg/ml to about 35 pg/ml, YKL-40 in the range of about 32 ng/ml to about 50 ng/ml, ICAM-1 in the range of about 200 ng/ml to about 320 ng/ml, VCAM-1 in the range of about 720 ng/ml to about 1065 ng/ml, NfL in the range of about 0.5 pg/ml to about 2.5 pg/ml, A1AT in the range of about 250 ng/ml to about 500 ng/ml, TTR in the range of about 3 μg/ml to about 10 μg/ml, Neurogranin in the range of about 4.5 pg/ml to about 7 pg/ml, and hFABP in the range of about 0.96 ng/ml to about 1.78 ng/ml, and
wherein detection that the biomarker is present in the reference value or amount range is indicative that the subject is afflicted with mild cognitive impairment with probable early AD (MCIAD) equivalent to an MMSE score of about 26.5 to about 26.8.

43. The system of claim 39 wherein the binding agents are selected from the group consisting of antibodies, antigens, nanoparticles, aptamers, inhibitors, substrates, cofactors, coenzymes, lectins, nucleic acids, protein A, protein G, nonbiological ligands, boronates, triazine dyes, and metal-ion chelates.

44. The system of claim 43 further comprising a solid support to which the binding agents are attached, the solid support being selected from an assay selected from the group of assays consisting of a lateral flow immunochromatographic assay (LFA), an enzyme-linked immunosorbent assay (ELISA), an enzyme-linked fluorescence polarization immunoassay (FPIA), a homogeneous immunoassay, a quantitative point-of-care assay using determination of chemiluminescence, fluorescence, magnetic particles, or latex agglutination, a gel electrophoresis assay, a gas chromatograph-mass spectrometry (GC-MS) assay, a separation immunoassay, a heterogeneous immunoassay, a homogenous immunoassay, a latex agglutination assay, a western blot assay, and a biosensor assay.

45. The system of claim 44 wherein the measurement device provides a visual indication of the label.

46. The system of claim 45 wherein the visual indication is a fluorescent indication.

47. The system of claim 39 wherein the binding agents are selected for their capability to bind with a specific one of the biomarkers in the combination of biomarkers selected from the group consisting of
(1) BACE1 and NfL;
(2) NfL and IGFBP-2;
(3) BACE1 and IGFBP-2;
(4) NfL and hFABP;
(5) NfL and GSH;
(6) NfL and Interleukin 6 (IL-6);
(7) BACE1 and hFABP;
(8) BACE1 and GSH;
(9) BACE1 and IL-6;
(10) IGFBP-2 and hFABP;
(11) IGFBP-2 and GSH;
(12) IGFBP-2 and IL-6;
(13) IGFBP-2 and IGFBP-3; and
(14) IGFBP-2 and Neurogranin.

48. A kit for detecting salivary biomarkers indicative that a subject is afflicted with Alzheimer's disease (AD) or the severity of AD in the subject, the kit comprising:
(a) a solid support on which a plurality of binding agents have been affixed, directly or indirectly, capable of binding with one or more biomarker in a saliva sample obtained from the subject selected from the group consisting of; Insulin-like growth factor binding protein-2 (IGFBP-2), Insulin-like growth factor binding protein-3 (IGFBP-3), Beta-secretase 1 (BACE1), Reduced glutathione (GSH), TNF-related apoptosis-including ligand (TRAIL), Interleukin 6 (IL-6), Chitinase-3-like protein 1 (YKL-40), Intercellular Adhesion Molecule 1 (ICAM-1), Vascular cell adhesion protein 1 (VCAM-1), Neurofilament protein L (NfL), Alpha-1 antitrypsin (A1AT), Transthyretin (TTR), Neurogranin, and Human heart fatty acid binding protein (hFABP), and combinations thereof;
(b) a measurable label associated with the binding agents which provides a detectable complex indicative of the amount of the one or more detected biomarker in the saliva sample, wherein IGFBP-2 above about 2500 pg/ml, IGFBP-3 above about 1680 ng/ml, BACE1 above about 700 pg/ml, GSH less than about 1.7 μmol/l, TRAIL above about 3 ng/ml, IL-6 above about 20 pg/ml, YKL-40 above about 30 ng/ml, ICAM-1 above about 200 ng/ml, VCAM-1 above about 450 ng/ml, NfL above about 0.3 pg/ml, A1AT less than about 650 ng/ml, TTR less than about 10 μg/ml, Neurogranin above about 5 pg/ml, and/or hFABP above about 0.80 ng/ml, is indicative that the subject is afflicted with AD.

49. The kit of claim 48 wherein the binding agents are selected from the group consisting of antibodies, antigens, nanoparticles, aptamers, inhibitors, substrates, cofactors, coenzymes, lectins, nucleic acids, protein A, protein G, nonbiological ligands, boronates, triazine dyes, and metal-ion chelates.

50. The kit of claim 49 wherein the binding agents are secured to the solid support to thereby immobilize the bound biomarkers on the solid support.

51. The kit of claim 50 wherein the binding agents affixed to the solid support bind with a specific one of the biomarkers in the combination of biomarkers selected from the group consisting of
(1) BACE1 and NfL;
(2) NfL and IGFBP-2;
(3) BACE1 and IGFBP-2;
(4) NfL and hFABP;
(5) NfL and GSH;
(6) NfL and Interleukin 6;
(7) BACE-1 and hFABP;
(8) BACE-1 and GSH;
(9) BACE-1 and IL-6;
(10) IGFBP-2 and hFABP;
(11) IGFBP-2 and GSH;
(12) IGFBP-2 and IL-6;
(13) IGFBP-2 and IGFBP-3; and
(14) IGFBP-2 and Neurogranin.

52. The kit of claim 51 wherein the binding agents affixed to the solid support bind with a specific one of the biomarkers IGFBP-2, IGFBP-3, and BACE1.

53. The kit of claim 51 wherein the binding agents affixed to the solid support bind with a specific one of the biomarkers IGFBP-2, Neurogranin, and GSH.

54. The kit of claim 50 wherein the kit is selected from the group consisting of an enzyme-linked immunosorbent assay (ELISA) type, and a lateral flow immunochromatographic assay (LFA) type.

55. The kit of claim 54 further comprising instructions describing how to use the kit and interpret each visible indication.

56. A method for treatment of a subject afflicted with Alzheimer's disease (AD) comprising the steps of:
(a) testing a saliva sample from the subject for levels of one or more of a group of biomarkers consisting of Insulin-like growth factor binding protein-2 (IGFBP-2), Insulin-like growth factor binding protein-3 (IGFBP-3), Beta-secretase 1 (BACE1), Reduced glutathione (GSH), TNF-related apoptosis-including ligand (TRAIL), Interleukin 6 (IL-6), Chitinase-3-like protein 1 (YKL-40), Intercellular Adhesion Molecule 1 (ICAM-1), Vascular cell adhesion protein 1 (VCAM-1), Neurofilament protein L (NfL), Alpha-1 antitrypsin (A1AT), Transthyretin (TTR), Neurogranin, and Human heart fatty acid binding protein (hFABP); and
(b) determining that the saliva sample is positive for AD if the levels of the one or more biomarkers of the group meet one or more criteria in a group of test criteria consisting of
(1) IGFBP-2 above about 2500 pg/ml;
(2) IGFBP-3 above about 1680 ng/ml;
(3) BACE1 above about 700 pg/ml;
(4) GSH less than about 1.7 μmol/l;
(5) TRAIL above about 3 ng/ml;
(6) IL-6 above about 20 pg/ml;
(7) YKL-40 above about 30 ng/ml;
(8) ICAM-1 above about 200 ng/ml;
(9) VCAM-1 above about 450 ng/ml;
(10) NfL above about 0.3 pg/ml;
(11) A1AT less than about 650 ng/ml;
(12) TTR less than about 10 μg/ml;
(13) Neurogranin level above about 5 pg/ml; and
(14) hFABP above about 0.80 ng/ml;
(c) administering a drug or agent to the subject; and
(d) comparing changes in the test criteria over time responsive to the drug or agent to determine whether the subject has benefited from treatment with said agent or drug.

57. A method for detecting whether a subject who is asymptomatic of Alzheimer's disease (AD) is actually afflicted with AD comprising the steps of:
(a) testing a saliva sample from a subject who is asymptomatic of AD for levels of one or more of a group of biomarkers consisting of Insulin-like growth factor binding protein-2 (IGFBP-2), Insulin-like growth factor binding protein-3 (IGFBP-3), Beta-secretase 1 (BACE1), Reduced glutathione (GSH), TNF-related apoptosis-including ligand (TRAIL), Interleukin 6 (IL-6), Chitinase-3-like protein 1 (YKL-40), Intercellular Adhesion Molecule 1 (ICAM-1), Vascular cell adhesion protein 1 (VCAM-1), Neurofilament protein L (NfL), Alpha-1 antitrypsin (A1AT), Transthyretin (TTR), Neurogranin, and Human heart fatty acid binding protein (hFABP); and
(b) determining that the saliva sample is positive for AD if the levels of the one or more biomarkers of the group meet one or more criteria in a group of test criteria consisting of
(1) IGFBP-2 in the range of about 2600 pg/ml to about 3800 pg/ml;
(2) IGFBP-3 in the range of about 2100 ng/ml to about 3000 ng/ml;
(3) BACE1 in the range of about 800 pg/ml to about 1109 pg/ml;
(4) GSH in the range of about 1 μmol/l to about 1.6 μmol/l;
(5) TRAIL in the range of about 3 ng/ml to about 5.5 ng/ml;
(6) IL-6 in the range of about 25 pg/ml to about 36 pg/ml;
(7) YKL-40 in the range of about 33 ng/ml to about 50 ng/ml;
(8) ICAM-1 in the range of about 200 ng/ml to about 320 ng/ml;
(9) VCAM-1 in the range of about 720 ng/ml to about 1065 ng/ml;
(10) NfL in the range of about 0.5 pg/ml to about 2.5 pg/ml;
(11) A1AT in the range of about 250 ng/ml to about 500 ng/ml;
(12) TTR in the range of about less than about 3 μg/ml to about 10 μg/ml;
(13) Neurogranin in the range of about 4.5 pg/ml to about 7 pg/ml; and
(14) hFABP in the range of about 0.96 ng/ml to about 1.78 ng/ml,
wherein detection that the biomarker is present in the reference value or amount range is indicative that the subject is afflicted with mild cognitive impairment with probable early AD (MCIAD) equivalent to an MMSE score of about 26.5 to about 26.8.
